SEQUENCE LISTING (19 sequences)

SEQ ID NO: 1 - protein, 10 aa, synthetic construct
  REGION 1..10: note = Synthetic
SAGGGSGGGS

SEQ ID NO: 2 - protein, 10 aa, synthetic construct
  REGION 1..10: note = Synthetic
GSGSGSGSGG

SEQ ID NO: 3 - DNA, 1446 bp, synthetic construct
  misc_feature 1..1446: note = Synthetic
  source 1..1446: mol_type = other DNA
atggctttgcctgttactgcgcttcttttgcctttggcattgttgcttcacgccgccaggcccgagcagaagctgatcagcgaggaggacctggacatacagatgacgcaaacaacttccagtcttagcgctagcctgggggatcgagtcaccatatcttgcagggcgtctcaagacattagcaagtatctcaattggtatcaacagaaacctgatggaacagttaaacttctgatttaccacacgagtcgcctgcactccggtgtgccctccagattctccggctcaggaagtggaaccgactactctctcaccatctccaacctcgaacaagaagacatagctacatacttttgccaacaaggtaataccctcccctataccttcggtggaggcactaagctggagatcacagggagcacgtccgggtctggcaaaccggggagtggtgaggggtctacgaagggagaagtcaagcttcaggagtcaggacccggtcttgtagctcccagccaaagcctgtcagttacatgcacggtttccggtgtgtctttgccagattatggcgtatcttggattcgccaaccgcctagaaagggacttgagtggttgggtgtcatttggggatcagaaacaacttactataacagtgctcttaagtccaggttgactataatcaaggacaatagtaagtcccaagtttttctgaaaatgaattccctgcagacagatgacaccgctatctactactgtgccaagcactactattatgggggctcttatgctatggactattggggtcaggggacatcagttactgtttccagcgaaagcaagtatggtcctccctgccccccgtgcccaatgttctgggtgctcgtggtcgtaggaggcgtactcgcctgctattcattgctggttactgtagcctttattatcttctgggtcttaattaagaagaaggtggccaaaaagccgacaaataaggccccgcaccctaaacaagagccgcaagagataaatttcccagacgatttgcctgggagcaacacggcggccccggtgcaagagacactgcacgggtgtcaacccgtcacccaagaagacggaaaggaaagtcggatctccgtccaggagcgacagtccgccggagggggatcaggaggcgggtccgaaaggcccccacctgtgcccaatcccgattatgaaccaattcggaaaggccaaagggacctgtactcaggcctgaatcaacggggcagtggctcgggctcggggtccggcggatactttctgggcagattggtaccaagggggcgaggtgcggctgaggctgccacacggaaacagaggataacggaaaccgagtctccgtatcaggaacttcagggacagcggtccgatgtttacagtgacctcaacacccaaagaccgtactacaagtag

SEQ ID NO: 4 - protein, 481 aa, synthetic construct
  REGION 1..481: note = Synthetic
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVLIKKKVAKKPTNKAPHPKQEPQEINFPDDLPGSNTAAPVQETLHGCQPVTQEDGKESRISVQERQSAGGGSGGGSERPPPVPNPDYEPIRKGQRDLYSGLNQRGSGSGSGSGGYFLGRLVPRGRGAAEAATRKQRITETESPYQELQGQRSDVYSDLNTQRPYYK

SEQ ID NO: 5 - DNA, 1590 bp, synthetic construct
  misc_feature 1..1590: note = Synthetic
  source 1..1590: mol_type = other DNA
atggctttgcctgttactgcgcttcttttgcctttggcattgttgcttcacgccgccaggcccgagcagaagctgatcagcgaggaggacctggacatacagatgacgcaaacaacttccagtcttagcgctagcctgggggatcgagtcaccatatcttgcagggcgtctcaagacattagcaagtatctcaattggtatcaacagaaacctgatggaacagttaaacttctgatttaccacacgagtcgcctgcactccggtgtgccctccagattctccggctcaggaagtggaaccgactactctctcaccatctccaacctcgaacaagaagacatagctacatacttttgccaacaaggtaataccctcccctataccttcggtggaggcactaagctggagatcacagggagcacgtccgggtctggcaaaccggggagtggtgaggggtctacgaagggagaagtcaagcttcaggagtcaggacccggtcttgtagctcccagccaaagcctgtcagttacatgcacggtttccggtgtgtctttgccagattatggcgtatcttggattcgccaaccgcctagaaagggacttgagtggttgggtgtcatttggggatcagaaacaacttactataacagtgctcttaagtccaggttgactataatcaaggacaatagtaagtcccaagtttttctgaaaatgaattccctgcagacagatgacaccgctatctactactgtgccaagcactactattatgggggctcttatgctatggactattggggtcaggggacatcagttactgtttccagcgaaagcaagtatggtcctccctgccccccgtgcccaatgttctgggtgctcgtggtcgtaggaggcgtactcgcctgctattcattgctggttactgtagcctttattatcttctgggtcttaattaagaggttgaagattcaggtccgcaaagcggcaataacgagctacgaaaagtccgacggcgtttatacgggtcttagcaccaggaaccaagagacctatgaaacattgaaacatgaaaaacccccccaatccgccggagggggatcaggaggcgggtcctggagacggaagagaaaggagaagcaatccgaaacttctcccaaggagttcctcaccatttacgaagatgtaaaggacctgaaaaccagacggaatcacgagcaagaacagaccttccctggcggcgggtcaactatctactcaatgatccagagtcaaagttctgctccaactagccaggagccggcgtacacgctttacagcctcattcaacctagccgcaaaagcggcagcaggaagagaaatcacagtccctcattcaacagtacaatctatgaggtgattggcaagtctcaaccaaaagcccagaaccctgcgcgactttccaggaaggaactcgagaacttcgacgtgtactccggcagtggctcgggctcggggtccggcggagaaaggcccccacctgtgcccaatcccgattatgaaccaattcggaaaggccaaagggacctgtactcaggcctgaatcaacggtag

SEQ ID NO: 6 - protein, 529 aa, synthetic construct
  REGION 1..529: note = Synthetic
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVLIKRLKIQVRKAAITSYEKSDGVYTGLSTRNQETYETLKHEKPPQSAGGGSGGGSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGSGSGSGSGGERPPPVPNPDYEPIRKGQRDLYSGLNQR

SEQ ID NO: 7 - DNA, 1359 bp, synthetic construct
  misc_feature 1..1359: note = Synthetic
  source 1..1359: mol_type = other DNA
atggctttgcctgttactgcgcttcttttgcctttggcattgttgcttcacgccgccaggcccgagcagaagctgatcagcgaggaggacctggacatacagatgacgcaaacaacttccagtcttagcgctagcctgggggatcgagtcaccatatcttgcagggcgtctcaagacattagcaagtatctcaattggtatcaacagaaacctgatggaacagttaaacttctgatttaccacacgagtcgcctgcactccggtgtgccctccagattctccggctcaggaagtggaaccgactactctctcaccatctccaacctcgaacaagaagacatagctacatacttttgccaacaaggtaataccctcccctataccttcggtggaggcactaagctggagatcacagggagcacgtccgggtctggcaaaccggggagtggtgaggggtctacgaagggagaagtcaagcttcaggagtcaggacccggtcttgtagctcccagccaaagcctgtcagttacatgcacggtttccggtgtgtctttgccagattatggcgtatcttggattcgccaaccgcctagaaagggacttgagtggttgggtgtcatttggggatcagaaacaacttactataacagtgctcttaagtccaggttgactataatcaaggacaatagtaagtcccaagtttttctgaaaatgaattccctgcagacagatgacaccgctatctactactgtgccaagcactactattatgggggctcttatgctatggactattggggtcaggggacatcagttactgtttccagcgaaagcaagtatggtcctccctgccccccgtgcccaatgttctgggtgctcgtggtcgtaggaggcgtactcgcctgctattcattgctggttactgtagcctttattatcttctgggtcttaattaagaggttgaagattcaggtccgcaaagcggcaataacgagctacgaaaagtccgacggcgtttatacgggtcttagcaccaggaaccaagagacctatgaaacattgaaacatgaaaaacccccccaatccgccggagggggatcaggaggcgggtccgcactctatctcctcagacgggatcaacgactcccgcctgacgcccacaaaccacctggtggaggttcctttcgcacaccgattcaggaagaacaggcagacgctcattctactctcgcaaaaatcggcagtggctcgggctcggggtccggcggagaacgacggcgcggcaagggacatgatggtctgtaccaaggtctctccacagcaacgaaggatacttacgacgctttgcacatgcaatag

SEQ ID NO: 8 - protein, 451 aa, synthetic construct
  REGION 1..451: note = Synthetic
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVLIKRLKIQVRKAAITSYEKSDGVYTGLSTRNQETYETLKHEKPPQSAGGGSGGGSALYLLRRDQRLPPDAHKPPGGGSFRTPIQEEQADAHSTLAKIGSGSGSGSGGERRRGKGHDGLYQGLSTATKDTYDALHM

SEQ ID NO: 9 - protein, 37 aa, unidentified
  REGION 1..37: note = Human herpesvirus 8 type P K1 (K1_HHV8P)
HCQKQSDSNKTVPQQLRDYYSLHDLCTEDYTQPVDWY

SEQ ID NO: 10 - protein, 45 aa, unidentified
  REGION 1..45: note = Epstein-Barr virus (strain B95-8) LMP2 (LMP2_EBVB9)
HSDYQPLGTQDQSLYLGLRCCRYCCYYCLTLESEERPPTPYRNTV

SEQ ID NO: 11 - protein, 45 aa, unidentified
  REGION 1..45: note = Bovine leukemia virus (ENV_BLV)
APHFPEISFPPKPDSDYQALLPSAPEIYSHLSPTKPDYINLRPCP

SEQ ID NO: 12 - protein, 24 aa, unidentified
  REGION 1..24: note = Mouse mammary tumor virus (strain C3H) (ENV_MMTVC)
SAYDYAAIIVKRPPYVLLPVDIGD

SEQ ID NO: 13 - protein, 171 aa, unidentified
  REGION 1..171: note = Rhesus monkey rhadinovirus H26-95 R1 (R1_RRV)
RCNENSESSTNSYASQTSYIQPSHNQRSNTNECSRHTYRNAHQEESIEELPNQHTSETDSCCQLVLLEVKNVAYDGPQENTINEVMEQYDDVVVENIEQTSYEDNVEHMDYSDTINPNFNYYSGLILEEVDEVFYNELENQYHGLILENLDHNEYNHLNELNMIEQYDWLE

SEQ ID NO: 14 - protein, 16 aa, unidentified
  REGION 1..16: note = African horse sickness virus (VP7_AHSV)
EYLLLVASLADVYAAL

SEQ ID NO: 15 - protein, 86 aa, unidentified
  REGION 1..86: note = IL-2RG
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 16 - protein, 286 aa, unidentified
  REGION 1..286: note = IL-2RB
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 17 - protein, 195 aa, unidentified
  REGION 1..195: note = IL-7R
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 18 - protein, 230 aa, unidentified
  REGION 1..230: note = IL-9R
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 19 - protein, 285 aa, unidentified
  REGION 1..285: note = IL-21R
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

(19) United States
(12) Patent Application Publication
(10) Pub. No.: US 2024/0190988 A1
(43) Pub. Date: Jun. 13, 2024

(54) NOVEL CHIMERIC ANTIGEN RECEPTORS AND LIBRARIES

(71) Applicant: Massachusetts Institute of Technology, Cambridge, MA (US)

(72) Inventors: Michael Birnbaum, Arlington, MA (US); Taeyoon Kyung, Chelsea, MA (US)

(73) Assignee: Massachusetts Institute of Technology, Cambridge, MA (US)

(21) Appl. No.: 18/505,606

(22) Filed: Nov. 9, 2023

Related U.S. Application Data
(62) Division of application No. 16/788,255, filed on Feb. 11, 2020, now abandoned.
(60) Provisional application No. 62/832,816, filed on Apr. 11, 2019.

(51) Int. Cl. C07K 16/30; C12N 5/0783; C12N 15/10

(52) U.S. Cl. CPC C07K 16/30; C12N 5/0636; C12N 15/1093; C07K 2317/24; C07K 2319/02; C07K 2319/03; C07K 2319/033; C07K 2319/30

(57) ABSTRACT

Provided herein are chimeric antigen receptor (CAR) viral libraries and methods of making the same. In some embodiments, the CAR comprises an intracellular domain (ICD) with at least one immune activation signaling domain, one co-stimulatory domain, and one or more inhibitory signaling domain or signaling domain from non-T cell lineages. In some embodiments, the signaling domains of the ICD are joined by distinct linkers of 10 amino acids. In some embodiments, the CARs contain a 18-nucleotide barcode in the 3′ untranslated region. Also provided herein, are CAR cell libraries and methods of making the same.

### RELATED APPLICATIONS

This application is a divisional of U.S. application Ser. No. 16/788,255, filed Feb. 11, 2020, which claims priority under 35 U.S.C. § 119(e) to U.S. Provisional Application Ser. No. 62/832,816, filed Apr. 11, 2019, the entire contents of each of which are incorporated by reference herein.

### REFERENCE TO AN ELECTRONIC SEQUENCE LISTING

The contents of the electronic sequence listing (M065670467US03-SEQ-HCL.xml; Size: 31,792 bytes; and Date of Creation: Nov. 8, 2023) is herein incorporated by reference in its entirety.

### GOVERNMENT RIGHTS

This invention was made with government support under CA014051 awarded by the National Institutes of Health. The government has certain rights in the invention.

### BACKGROUND

Cells of the immune system such as T lymphocytes (T cells) recognize and interact with specific antigens through receptors or receptor complexes which, upon recognition or an interaction with such antigens, cause activation of the cell. An example of such a receptor is the antigen-specific T lymphocyte receptor (TCR) which is expressed on the surface of T lymphocytes. The TCR along with a transmembrane domain and intracellular domain form the TCR complex. These functions (antigen-binding, signaling, and stimulatory) when reduced by genetic recombination methods to a single polypeptide chain are generally referred to as a Chimeric Antigen Receptor (CAR). T cells engineered to express CARs (CAR-T cells) are interesting targets for research and development due to their efficacy, user-defined specificity, and potential as a general strategy for treating a wide variety of diseases.

The molecular architecture of a CAR can be separated into three components: an extracellular ligand-recognizing domain (typically, although not exclusively, an scFv), a spacer and transmembrane domain (borrowed from other proteins such as antibody hinge regions and CD28 respectively), and intracellular immune signaling motifs (almost always the intracellular domain (ICD) of the TCR signaling component CD3ζ combined with one or more T cell costimulatory domains, such CD28 or 4-1BB). CAR-T cells recognizing the B cell surface protein CD19 have shown remarkable success in eliminating B cell lymphomas and preventing recurrence of disease, thereby prolonging patient life. These treatments represent the first engineered cell therapeutics to receive FDA approval. However, the success seen with CD19-targeting CARs has yet to translate to other cancers, most notably solid tumors, because of both lack of efficacy and serious morbidities and mortalities.

### SUMMARY

In some aspects, the invention is a retroviral library, comprising a plurality of retroviruses, wherein each retrovirus comprises a unique CAR, wherein the CAR is comprised of an extracellular domain, a transmembrane domain, and an intracellular domain (ICD), wherein the retroviral library comprises at least 500,000 distinct unique CARs. In some embodiments, the retrovirus is a lentivirus.

In other aspects, a high complexity CAR cell library is provided. The cell library comprises a plurality of cells, each of the plurality of cells comprising a unique CAR, wherein the CAR is comprised of an extracellular domain, a transmembrane domain, and an intracellular domain (ICD), wherein the CAR cell library comprises at least 500,000 distinct unique CARs.

In some embodiments, at least one of the unique CARs comprises a signaling domain from a non-T cell lineage. In other embodiments at least 50% of the unique CARs comprise a signaling domain from a non-T cell lineage. In some embodiments, the unique CARs comprises a signaling domain from a non-T cell lineage. In some embodiments, the signaling domain from a non-T cell lineage is a B cell signaling domain. In other embodiments the signaling domain from a non-T cell lineage is a macrophage signaling domain. In some embodiments, the signaling domain from a non-T cell lineage is selected from the group consisting of: CD79A, CD79B, FCER1G, CD19, CD40, KIR3DL1, KIR3DL2, KIR2DL3, KIR2DL4, KIR2DL5, KIR3DL1, KIR3DL2, KIR3DL3, SIRPA, FCRL1, FCRL2, FCRL3, FCRL4, FCRL5, FCRL6, FCGR1A, FCGR2A, FCGR2B, FCGR3A, TLR1, TLR2, TLR3, TLR4, TLR5, TLR6, TLR7, TLR8, TLR9, TLR10, PILRB, NCR1, NCR2, NCR3, NKG2A, NKG2C, NKG2D, CD22.

In some embodiments, each unique CAR is comprised of at least one or two ICD modules, wherein the identity and/or arrangement of the at least one or two ICD modules is different from the identity and/or arrangement of the at least one or two ICD modules in at least 50% of the other unique CARs in the cell library in some embodiment. In some embodiments, each unique CAR is comprised of at least three ICD modules, wherein the identity and/or arrangement of the at least three ICD modules is different from the identity and/or arrangement of the at least three ICD modules in at least 50% of the other unique CARs in the cell library. In some embodiments, the at least three ICD modules are selected from the group consisting of an immune activation signaling domain, a co-stimulatory domain, an inhibitory signaling domain and a signaling domain from non-T cell lineage. In some embodiments, the immune activation domain is a human immune activation domain. In other embodiments the immune activation domain is a virally encoded immune activation domain. In other embodiments the ICD comprises a growth factor receptor. The growth factor receptor in some embodiments is IL-2Rβ or IL-2Rγ.

In some embodiments, each unique CAR has at least 2 ICD modules, wherein at least 50 distinct modules are represented in the CAR library. In other embodiments each unique CAR has at least 2 ICD modules, wherein at least 75 distinct modules are represented in the library. In other embodiments each unique CAR has at least 2 ICD modules, wherein at least 85 distinct modules are represented in the CAR library.

In some embodiments, each unique CAR does not include a reporter protein.

In some embodiments, each cell comprises a nucleic acid encoding the unique CAR, wherein the nucleic acid comprises nucleotides coding for the unique CAR and a unique nucleic acid barcode that is specific for the unique CAR.

In other embodiments the library comprises at least 1×10⁶ distinct unique CARs.

In some embodiments, the cells of the CAR cell library are T cells such as primary T cells and T cell lines.

In some embodiments, the library comprises at least 2 or 3 different extracellular domains. In other embodiments the library comprises at least 2 or 3 different transmembrane domains.

In some embodiments, each unique CAR comprises a linker between one or more domains. In some embodiments, the linker is a sequence of 10 amino acids. In other embodiments the CAR has 3 or more signaling domains and the linkers between each signaling domain are distinct. In some embodiments, at least one of the linker amino acid sequences between signaling domains comprises: SAGGGSGGGS (SEQ ID NO:1) or GSGSGSGSGG (SEQ ID NO:2).

In other aspects, the invention is a method for preparing a CAR viral library, comprising:
i) providing a sample of ICD signaling domains, which sample includes an immune activation signaling domain, a co-stimulatory domain, and at least one signaling domain selected from the following signaling domains: inhibitory signaling domains and signaling domains from non-T cell lineages;
ii) assembling the CAR by overlap-extension polymerase chain reaction (PCR) and Gibson Assembly of the ICD signaling domains, a transmembrane domain, and an extracellular domain; and
iii) insertion of the CAR in a retroviral vector.

In some embodiments, the method further comprising iv) transducing cells with the retroviral vector after iii). In some embodiments, step ii) further comprises linking the signaling domains by a linker sequence of 10 amino acids.

In some embodiments, the CAR has 3 or more signaling domains and the linkers between each signaling domain are distinct. In some embodiments, the at least one of the linker sequences between signaling domains comprises: SAGGGSGGGS (SEQ ID NO:1) or GSGSGSGSGG (SEQ ID NO:2). In some embodiments, the CAR has 3 signaling domains and the linkers consist of SEQ ID NO:1 and SEQ ID NO:2.

In some aspects, the invention is a method of screening a CAR-T cell library, the method comprising: activating CAR-T cells; sorting the activated CAR-T cells by FACS by a predetermined intensity; and repeating the process with CAR-T cells of step ii) at least one additional time.

The invention in some aspects, is a CAR, comprising: an extracellular domain; a transmembrane domain; and at least a first intracellular domain (ICD) and a second ICD, wherein, the first ICD is linked to the second ICD by a linker comprising at least 10 amino acids.

In some aspects, the invention is a nucleic acid, comprising a coding region that encodes the CAR as described herein.

In some embodiments, the nucleic acid sequence has at least 70% sequence identity to (SEQ ID NO:3). In some embodiments, the nucleic acid sequence has at least 80% sequence identity to (SEQ ID NO:3). In some embodiments, in the nucleic acid sequence has at least 85% sequence identity to (SEQ ID NO:3). In some embodiments, the nucleic acid sequence has at least 90% sequence identity to (SEQ ID NO:3). In some embodiments, the nucleic acid sequence has at least 95% sequence identity to (SEQ ID NO:3). In some embodiments, the nucleic acid sequence has at least 99% sequence identity to (SEQ ID NO:3). In some embodiments, the nucleic acid sequence has at least 70% sequence identity to (SEQ ID NO:5). In some embodiments, the nucleic acid sequence has at least 80% sequence identity to (SEQ ID NO:5). In some embodiments, nucleic acid sequence has at least 85% sequence identity to (SEQ ID NO:5). In some embodiments, the nucleic acid sequence has at least 90% sequence identity to (SEQ ID NO:5). In some embodiments, the nucleic acid sequence has at least 95% sequence identity to (SEQ ID NO:5). In some embodiments, the nucleic acid sequence has at least 99% sequence identity to (SEQ ID NO:5). In some embodiments, the nucleic acid sequence has at least 70% sequence identity to (SEQ ID NO:7). In some embodiments, the nucleic acid sequence has at least 80% sequence identity to (SEQ ID NO:7). In some embodiments, the nucleic acid sequence has at least 85% sequence identity to (SEQ ID NO:7). In some embodiments, the nucleic acid sequence has at least 90% sequence identity to (SEQ ID NO:7). In some embodiments, the nucleic acid sequence has at least 95% sequence identity to (SEQ ID NO:7). In some embodiments, the nucleic acid sequence has at least 99% sequence identity to (SEQ ID NO:7).

In other aspects, the invention is a polypeptide, comprising an amino acid sequence translated from the coding region that encodes the CAR as described herein. In some embodiments, the amino acid sequence has at least 70% sequence identity to (SEQ ID NO:4). In some embodiments, the amino acid sequence has at least 80% sequence identity to (SEQ ID NO:4). In some embodiments, the amino acid sequence has at least 85% sequence identity to (SEQ ID NO:4). In some embodiments, the amino acid sequence has at least 90% sequence identity to (SEQ ID NO:4). In some embodiments, the amino acid sequence has at least 95% sequence identity to (SEQ ID NO:4). In some embodiments, the amino acid sequence has at least 99% sequence identity to (SEQ ID NO:4). In some embodiments, the amino acid sequence has at least 70% sequence identity to (SEQ ID NO:6). In some embodiments, the amino acid sequence has at least 80% sequence identity to (SEQ ID NO:6). In some embodiments, the amino acid sequence has at least 85% sequence identity to (SEQ ID NO:6). In some embodiments, the amino acid sequence has at least 90% sequence identity to (SEQ ID NO:6). In some embodiments, the amino acid sequence has at least 95% sequence identity to (SEQ ID NO:6). In some embodiments, the amino acid sequence has at least 99% sequence identity to (SEQ ID NO:6). In some embodiments, the amino acid sequence has at least 70% sequence identity to (SEQ ID NO:8). In some embodiments, the amino acid sequence has at least 80% sequence identity to (SEQ ID NO:8). In some embodiments, the amino acid sequence has at least 85% sequence identity to (SEQ ID NO:8). In some embodiments, the amino acid sequence has at least 90% sequence identity to (SEQ ID NO:8). In some embodiments, the amino acid sequence has at least 95% sequence identity to (SEQ ID NO:8). In some embodiments, the amino acid sequence has at least 99% sequence identity to (SEQ ID NO:8).

In yet other embodiments the nucleic acid further comprises a 18-nucleotide long barcode in a 3′ untranslated region (3′-UTR).

In some aspects, the invention is a CAR, comprising: an extracellular domain; a transmembrane domain; and an intracellular domain (ICD) comprised of three linked modules, wherein the three linked modules are CD40-CD3zITAM3-DAP12.

In other aspects, the invention is a CAR, comprising: an extracellular domain; a transmembrane domain; and an intracellular domain (ICD) comprised of three linked modules, wherein the three linked modules are FCER1G-2B4-CD3zITAM3.

In yet other aspects, the invention is a CAR, comprising: an extracellular domain; a transmembrane domain; and an intracellular domain (ICD) comprised of three linked modules, wherein the three linked modules are FCER1G-OX40-CD3zITAM.

In yet other aspects, the invention is a CAR, comprising: an extracellular domain; a transmembrane domain; and an intracellular domain (ICD) comprised of three linked modules, wherein the three linked modules are CD40-CD3eITAM-DAP12.

In yet other aspects, the invention is a CAR, comprising: an extracellular domain; a transmembrane domain; and an intracellular domain (ICD) comprised of three linked modules, wherein the three linked modules are FCER1G-2B4-CD3eITAM.

In yet other aspects, the invention is a CAR, comprising: an extracellular domain; a transmembrane domain; and an intracellular domain (ICD) comprised of three linked modules, wherein the three linked modules are FCER1G-OX40-CD3zITAM3.

In yet other aspects, the invention is a CAR, comprising: an extracellular domain; a transmembrane domain; and an intracellular domain (ICD) comprised of three linked modules, wherein the three linked modules are PILRB-FCER1G-CD3zITAM3.

In yet other aspects, the invention is a CAR, comprising: an extracellular domain; a transmembrane domain; and an intracellular domain (ICD) comprised of three linked modules, wherein the three linked modules are CD3zITAM3-CD3d-CD4.

In yet other aspects, the invention is a CAR, comprising: an extracellular domain; a transmembrane domain; and an intracellular domain (ICD) comprised of three linked modules, wherein the three linked modules are CD79a-CD79aITAM-CD4.

In yet other embodiments or aspects the invention encompasses any of the paragraphs listed under the heading “other embodiments.”

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

FIGS. 1A-IC show the structure, characterization, and elements of an anti-CD19 CAR design. FIG. 1A: shows the design of a modular CAR designed molecule and resulting ICD diversity. FIG. 1B: shows a non-limiting list of ICD components, included are immune activation, co-stimulatory, inhibitory, and other immune signaling molecules. FIG. 1C: shows a DNA library design and good agreement with range of possible sizes (right) and experimental result (left).

FIGS. 2A-2C show a procedural diagram and schematic representation of the workflow for creating and employing the CAR-T cell library. The experimental design of the process, including CAR library construction incorporation via lentivirus is shown in the left panel (FIG. 2A). The selection strategies of iterative selection and sorting based on traits of interest is shown in the middle panel (FIG. 2B). The sequencing and quantification process using both an long amplicon based reads for identification as well as sequencing with deep reads for quantification is shown in the right panel (FIG. 2C).

FIG. 3A-3B show the library selection diversity results. FIG. 3A: After exposure to CD19, each round of FACS sorting was sorted by upregulation of CD69. FIG. 3B: Frequency of top 100 barcode sequences is shown.

FIG. 4 shows the frequency of ICD signaling domains after iterative selection for upregulation of CD69+ after exposure to CD19.

FIG. 5 shows a canonical 2^{nd} generation CAR construct along with 6 CAR variants resulting from the library and/or methods in the instant disclosure.

FIGS. 6A-6D show altered PD1 and CD69 expression at both saturating activation (top; FIGS. 6A-6B) and basal activity (bottom; FIGS. 6C-6D).

FIG. 7 shows increased interferon gamma (IFN-γ) production of CAR-T cells from the library and/or methods herein compared to 2^{nd} generation CAR-T cells (LCAR, which consists of the signaling domains 4-1BB-CD3-zeta).

FIGS. 8A-8B show the increased proliferation (FIG. 8A) and altered exhaustion markers (including PD-1, LAG-3, and TIM-3; FIG. 8B) for CAR-T cells from the library and/or methods herein compared to LCAR. Altered expression of canonical T cell exhaustion markers. Columns (as read left to right): 1-4 show PD-1 expression; 5-8 show Tim-3 expression; and 9-12 show LAG-3 expression.

FIGS. 9A-9B show the increased cytotoxicity of CAR-T cell variants from the library and/or methods herein compared to LCAR. Two cell lines are shown (Raji: FIG. 9A; NALM6: FIG. 9B) with cell killing percent on the y-axis and the effector target (E:T) ratio shown on the x-axis.

FIGS. 10A-JOB show the effect of CAR-T cells Var1 and 3 on NALM6 cell killing using a Luciferase assay, where CAR-T cells were co-incubated with NALM6-luciferase for 6 h before luciferase assay. The left panel (FIG. 10A) shows a linear depiction of the data (with cell killing percent on the y-axis and the effector target (E:T) ratio shown on the x-axis) and the right panel (FIG. 10B) assesses percent specific lysis for LCAR vs Var1 and Var3.

FIG. 11 shows the effect of CAR-T cells Var1 and 3 on NALM6 cell killing using a Luciferase assay where CAR-T cells were co-incubated with NALM6-luciferase for 3 h before luciferase assay, with cell killing percent on the y-axis and the effector target (E:T) ratio shown on the x-axis.

FIGS. 12A-12B show a comparison of basal signaling states based on CD69 and PD-1 level in unstimulated CD8+ T cells from 3-4 different donors. Var1 (CD40, CD3e ITAM, and DAP12) shows a lower degree of basal signaling assessed by activation markers, CD69 (FIG. 12A) and PD-1 (FIG. 12B), over other CARs. Prior literature has shown that CARs with higher basal signaling states lead to lower efficacy in the clearance of tumors in vivo. Mock: Cells treated identically to CAR-transduced cells but that do not express a CAR construct; LCAR chimeric antigen receptor encoding the 4-1BB and CD3Z signaling domains as currently used in the clinic); DCAR, an LCAR construct with each Tyrosine that is phosphorylated via signaling mutated to Phenylalanine, creating a signaling-inactive CAR variant; and Var3 (FCER1G-OX40-CD3z ITAM 3).

FIGS. 13A-13D show differential cytokine and chemokine programs shown by Var1 and Var3 compared to LCAR in CD4+(FIG. 13C: Var 1 in CD4+; FIG. 13D: Var 3 in CD4+) or CD8+T (FIG. 13A: Var 1 in CD8+; FIG. 13B: Var 3 in CD8+) cells. Var1 and Var3 show elevated levels of secreted anti-tumor cytokines and chemokines over LCAR, such as MIP1a, FLT3L, IL-12p70, TNF-a, GM-CSF, and IL-2. CAR-T cells were co-cultured with NALM6 cells at effector:target ratio of 1:1 for 24 h prior to 41-plex Luminex assay.

FIG. 14 shows the killing capacity of CAR-T cells at increasing effector:target ratios. Var1 in CD8+ T cells shows results at controlling high tumor burden compared to that of LCAR. CAR-T cells were co-cultured with NALM6 cells at designated effector:target ratio for 24 h prior to luciferase assay.

FIGS. 15A-15D show measurement of IL-2 and IFN-γ levels, two major anti-tumor cytokines of T cells, of CD8+(FIG. 15A: IL-2 1 in CD8+; FIG. 15B: IFNγ (IFN-gamma) in CD8+) or CD4+(FIG. 15C: IL-2 1 in CD4+; FIG. 15D: IFNγ (IFN-gamma) in CD4+) CAR-T cells challenged with NALM6 cells at designated effector:target ratio. TFNγ secretion level of Var1 is significantly higher than that of other types of CARs across different amount of tumor burden in both CD4+ and CD8+ T cells. CAR-T cells were co-cultured with NALM6 cells for 24 h prior to ELISA assay. Columns (as read from left to right) at each E:T ratio: 1—Mock; 2—DCAR; 3—LCAR; 4—Var 1; and 5—Var 3. Note, in panel FIG. 15C, for E:T ratios 1:1, 1:2, and 1:5, only columns (as read from left to right) 3-5 show secretion. Note, in panel FIG. 15D, for E:T ratios 1:1, 1:2, and 1:5, only columns (as read from left to right) 4-5 show secretion and E:T ratio 1:10 only secretion for columns 2-5.

FIGS. 16A-16D show CAR-T cell proliferation and tumor control in CD8+ or CD4+ CAR-T cells undergoing repetitive challenge with NALM6 cells (CD8+: FIG. 16A and FIG. 16C; CD4+: FIG. 16B and FIG. 16D) every 48 h to maintain effector:target ratio of 1:2. Var1 and Var3 (FIG. 16B and FIG. 16D) in CD4+ T cells show better proliferation and tumor control compared to those of LCAR. Proliferation and tumor control were assessed by counting absolute cell numbers in the culture across 8 days.

FIGS. 17A-17B show exhaustion marker staining on CD4+(FIG. 17A) or CD8+(FIG. 17B) CAR-T cells at day 10 of repetitive challenge assay. CAR-T cells were co-cultured with NALM6 cells and effector:target ratio of 1:2 was maintained throughout by adding target cells every 48 h. High expression of PD-1, TIM3, and LAG3 exhaustion markers in dysfunctional T cells is a hallmark of exhausted T cells. In this case, differential expression levels of exhaustion markers among CAR-T cells, Var 1, and Var 3, indicating that it is challenging to assess which type of CAR is more exhausted relative to others in this type of assay. Columns (as read from left to right) for each marker (i.e., PD-1, TIM3, and LAG3): 1—LCAR; 2—Var 1; and 3—Var 3.

### DETAILED DESCRIPTION

While both present and prospective engineered T cell approaches have promise, they do not address several fundamental limitations to creating the next generation of engineered cell therapeutics. At present, most described methods for generating novel CAR constructs for use in T cells or other immune cells relies either upon conservative iteration of previously described designs, some hypothesis-based alteration of CARs based upon literature data, or serendipity. The methods of the invention involve a library-based system to address some of these shortfalls.

The libraries produced according to the invention are highly diverse complex libraries generated in retroviral vectors and used to produce cellular libraries expressing multiple unique CARs. CAR libraries described in the prior art have been limited in diversity because fewer domains were altered and combined. For instance a relatively recent CAR based library described by Doung C et al. (Engineering T Cell Function Using Chimeric Antigen Receptors Identified Using a DNA Library Approach. PLoS ONE 8(5) 2013) consists of combinations of only 14 ICDs and includes a very wide range for the number of possible ICDs per construct. These together lead to constructs that can have extensive repeats, limiting their likelihood of signaling and folding. Additionally, the overall library size or diversity was ˜10⁴. In contrast, the libraries generated according to the invention have in some embodiments greater than 50 and even 85 possible domains or modules for assembly of the ICD leading to significantly higher diversity on the order of ˜10⁶. The prior art methods were not able to achieve the creation or analysis of the library data. For instance, the prior art samples were not deep sequenced in any way, leaving analysis to only ˜100 clones. The data is fully accessible using the unique selection and analytical methods disclosed herein.

The ability to design a library of such complexity is advantageous for a number of reasons. Based upon the data generated to date it appears that optimal CAR development will vary depending upon the purpose. It is expected that there will not be a single optimal combination of ICDs for CAR function. Instead, factors such as desired phenotype, solid vs. liquid tumor, affinity of the extracellular binding domain to its target, antigen density, and a host of other factors are likely to influence the needs of CAR function and thus the composition of the CAR ICD. These methods can be used to achieve that personalization or optimization of CAR tools.

Additionally, all present receptors rely upon only a few previously established signaling motif combinations (i.e., CD3ζ+CD28/OX-40/4-1BB ICDs) in the intracellular domain (ICD). It is through the work of the invention that it was found that there is no inherent reason why the handful of established ICD compositions in the art represent the optimal configurations. For example, T cell programs such as resistance to T cell exhaustion, target cell killing without systemic release of cytokines, or novel combinations of effector molecules may simultaneously be transformative for treatment and feasible as a cellular output, but not presently achievable due to limitations in antigen receptor design.

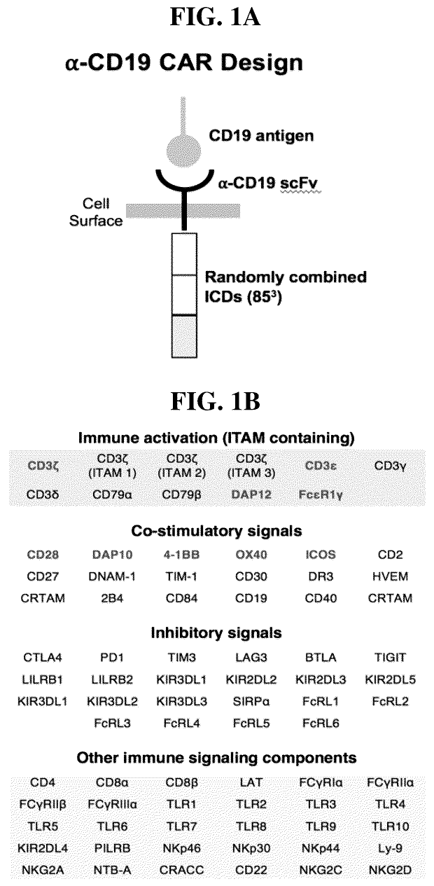

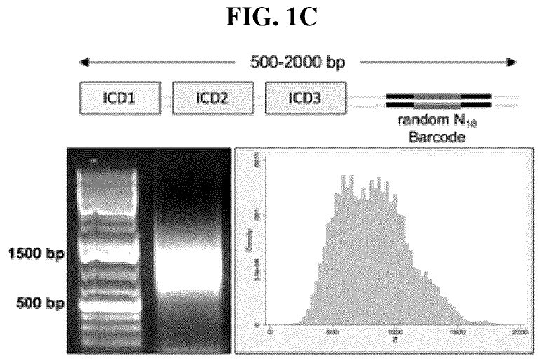

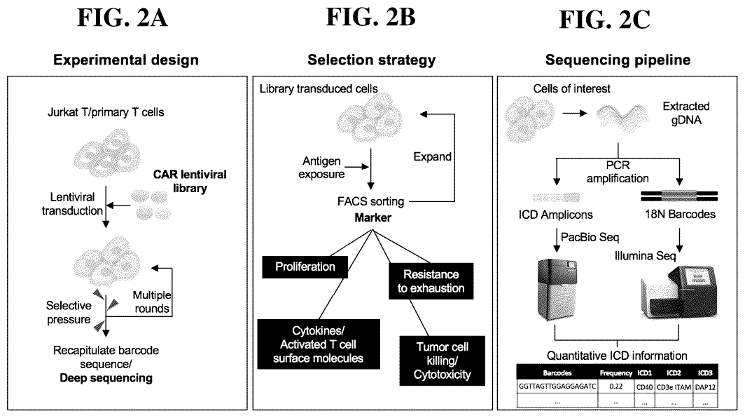

The development of the instant CAR viral and cell libraries, produces inherent diversity in the composition and amino acid sequence of CARs (see for instance, FIGS. 1A-1C, and 2). The libraries are first assembled into a plasmid library, and then used to create a lentiviral library. The lentiviruses are then transduced into T cells (either primary T cells or T cell lines), and these cells are sorted for an activity of interest. The selected T cells can then be sequenced to determine what CAR variant conferred the desired function of interest.

Accordingly, provided herein is a CAR viral library, which is highly complex and diverse, which utilizes signaling motifs (Modules) from a broad spectrum of cells. Also provided herein, are methods of constructing the viral library, as well as CAR cell libraries, and useful CAR sequences.

In one aspect of the instant disclosure, a high complexity retroviral library containing nucleic acids encoding for unique CARs is provided. Viral libraries are any collections of viral vectors which vary in the nucleic acid to be delivered to the host cell and which are used in a variety of manners, they are single preparations of many different viral vectors.

The retroviral vectors disclosed herein comprise one or more elements derived from a retroviral genome (naturally-occurring or modified) of a suitable species. Retroviruses include 7 families: alpharetrovirus (Avian leucosis virus), betaretrovirus (Mouse mammary tumor virus), gammaretrovirus (Murine leukemia virus), deltaretrovirus (Bovine leukemia virus), epsilonretrovirus (Walleye dermal sarcoma virus), lentivirus (Human immunodeficiency virus 1), and spumavirus (Human spumavirus). Six additional examples of retroviruses are provided in U.S. Pat. No. 7,901,671.

In some embodiments of the instant disclosure, lentivirus is used as the viral vector. Lentivirus is a genus of retroviruses that typically gives rise to slowly developing diseases due to their ability to incorporate into a host genome. Modified lentiviral genomes are useful as viral vectors for the delivery of a nucleic acids to a host cell. Host cells can be transfected with lentiviral vectors, and optionally additional vectors for expressing lentiviral packaging proteins (e.g., VSV-G, Rev, and Gag/Pol) to produce lentiviral particles in the culture medium.

Retroviral and lentiviral vectors are well known in the art and any suitable retrovirus can be used to construct the retroviral vector library as described herein. Non-limiting examples of retroviral vectors include lentiviral vectors, human immunodeficiency viral (HIV) vector, avian leucosis viral (ALV) vector, murine leukemia viral (MLV) vector, murine mammary tumor viral (MMTV) vector, murine stem cell virus, and human T cell leukemia viral (HTLV) vector. These retroviral vectors comprise proviral sequences from the corresponding retrovirus.

The retroviral vectors described herein may comprise the viral elements such as those described herein from one or more suitable retroviruses, which are RNA viruses with a single strand positive-sense RNA molecule. Retroviruses comprise a reverse transcriptase enzyme and an integrase enzyme. Upon entry into a target cell, retroviruses utilize their reverse transcriptase to transcribe their RNA molecule into a DNA molecule. Subsequently, the integrase enzyme is used to integrate the DNA molecule into the host cell genome. Upon integration into the host cell genome, the sequence from the retrovirus is referred to as a provirus (e.g., proviral sequence or provirus sequence). This efficient gene transfer mechanism has made retroviral vectors highly valuable tools in gene therapy, because they can be used for long term transgene expression in host cells. The retroviral vectors described herein may further comprise additional functional elements as known in the art to address safety concerns and/or to improve vector functions, such as packaging efficiency and/or viral titer. Additional information may be found in US20150316511 and WO2015/117027, the relevant disclosures of each of which are herein incorporated by reference for the purpose and subject matter referenced herein. Additional information for lentiviral vectors can be found in, e.g., WO2019/056015, the relevant disclosures of which are incorporated by reference herein for this particular purpose.

Retroviral vectors such as lentiviral vectors and gamma retroviral vectors provide an efficient means for carrying the genetic information of the plasmids, such as introducing new genes, into human and animal cells. Multiple generations of retroviral vector systems have been developed to minimize the safety considerations due to the pathogenicity of HIV-1, from which many vectors are derived. For example, third-generation, self-inactivating retroviral vectors have been used in clinical trials for introducing genes into host cells such as hematopoietic for treating various genetic disorders.

Many times viral vectors of viral libraries encode sequences of particular interest for the field of research for which the library was constructed. The viral vectors may encode for sequences of interest, whereby the sequences will facilitate protein production, which proteins may be useful by the host organism of the transduced cell or by the transduced cell itself. For example, the sequences may comprise sequences encoding for production of proteins useful as signaling components and complexes useful in activating or inhibiting cellular function. For example, cells of the immune system such as T lymphocytes (T cells) recognize and interact with specific antigens through receptors or receptor complexes which, upon recognition or an interaction with such antigens, cause activation of the cell. An example of such a receptor is the antigen-specific TCR which is expressed on the surface of T cells. The TCR in conjunction with the transmembrane domain (the receptor complex that connects the extracellular portion of the complex with the intracellular domain through the cellular membrane) and intracellular domain (the portion of the receptor complex that effect cellular response in the cytoplasm of the cell) form the TCR complex. The TCR complex functions (antigen-binding, signaling, and stimulatory) can be reduced by genetic recombination methods to a single protein chain, which is known as a CAR.

Viral libraries can vary in size from small libraries carrying nucleic acids from a few plasmids to hundreds of thousands, millions, or more plasmids. In some embodiments of the viral library of the instant disclosure, comprises at least 500,000 distinct viral plasmids.

The libraries of the invention include retroviral libraries and cellular libraries. A library is a synthetic (i.e., isolated, synthetically produced, free from components that are naturally found together in a cell, purified before being put into the library) collection of members having a common element and at least one distinct element. The library comprises a thousand or more (e.g., at least: 1,000; 2,000; 3,000; 4,000; 5,000; 10,000; 50,000; 100,000; 500,000; 600,000; 700,000; 800,000; 900,000; 1,000,000; 2,000,000; 3,000,000; 4,000,000; or more) members. The upper limit of the library size is defined by the combinatorics of domains or modules providing distinctness or diversity among the members. For instance, an upper limit may be 4,000,000 members. Thus, in some embodiments, the library is highly diverse, and includes at least 500,000 distinct members. The highly diverse library may have a diversity of 10⁶ or greater.

A retroviral library is a collection of retroviral vectors each vector including a nucleic acid encoding a unique protein such as a CAR. A cellular library is a collection of cells (having been transfected with a library of retroviral vectors) each cell including a unique protein such as a CAR.

In some embodiments, the libraries express or encode a collection of unique CARs. A “chimeric antigen receptor” or “CAR” as used herein refers to a fused protein comprising an extracellular domain capable of binding to an antigen or ligand, a transmembrane domain typically derived from a polypeptide different from a polypeptide from which the extracellular domain is derived, and at least one intracellular domain.

A unique CAR, as used in the context of a library, refers to a CAR polypeptide or a nucleic acid encoding a CAR polypeptide having an extracellular domain, a transmembrane domain, and an intracellular domain, wherein the amino acid sequence of the CAR polypeptide is distinct from each other CAR polypeptide in the library. A CAR polypeptide or nucleic acid is said to be distinct from each other member of the library when the CAR differs from the other members by at least one amino acid or nucleotide. In some instances, the CAR differs from the other members by at least one domain or module. The difference between unique CARs may be at the level of identity or arrangement or position. A set of unique CARs, for instance may have all of the same domains or modules, but those domains or modules are organized or arranged in a different order from one another. Such a set of unique CARs would be referred to as having differences in arrangement or position. Another set of unique CARs may have at least one different module or domain having a distinct amino acid sequence from the other CAR. Such a set is referred to as a set having a different identity.

A library of unique CARs as described herein may contain duplicate CARs. For instance, a retroviral library may include 2 or more copies of a nucleic acid encoding a unique CAR and a cellular library may contain 2 or more copies of a unique CAR. The duplicate copies of a unique CAR, however, are not included in the calculation of diversity. When a library is referred to as having members and each member being or encoding a unique CAR, such a library may also include duplicates.

As used herein, a “domain” is used interchangeably with the term “module” to mean one region in a polypeptide which is independent of other regions in the polypeptide, either functionally or structurally. The modules are described by name and or sequence. Exemplary sequences are provided herein. However, the claimed modules and structures are not limited to the exemplified sequences. The skilled artisan is familiar with extracellular domains, intracellular domains and transmembrane domains. Any such domains may be used in the constructs including naturally occurring versions of those domains, modified versions and synthetic versions. For instance the term CD3z refers to the zeta chain of CD3 and may include naturally occurring CD3z sequences as well as modified CD3z and synthetic CD3z sequences. Many sequences are included in publically available databases.

The “extracellular domain” means any oligopeptide or polypeptide that can bind to a certain antigen or ligand. It may be a receptor, typically, although not exclusively, a single chain variable fragment (scFv). As used herein, a “single chain variable fragment” or “scFv)” means a single chain polypeptide derived from an antibody which retains the ability to bind to an antigen. An example of the scFv includes an antibody polypeptide which is formed by a recombinant DNA technique and in which Fv regions of immunoglobulin heavy chain (H chain) and light chain (L chain) fragments are linked via a spacer sequence. Various methods for preparing an scFv are known to a person skilled in the art.

In some embodiments, the antigen or ligand is a tumor antigen or ligand associated with the surface of a tumor cell. The antigen or ligand may be, for instance, any one or more of CD19, CD20, BCMA, CD22, CD38, CD138, mesothelin, VEGFR-2, CD4, CD5, CD30, CD22, CD24, CD25, CD28, CD30, CD33, CD47, CD52, CD56, CD80, CD81, CD86, CD123, CD171, CD276, B7H4, CD133, EGFR, GPC3; PMSA, CD3, CEACAM6, c-Met, EGFRvIII, ErbB2/HER-2, ErbB3/HER3, ErbB4/HER-4, EphA2,10a, IGF1R, GD2, 0-acetyl GD2, 0-acetyl GD3, GHRHR, GHR, FLT1, KDR, FLT4, CD44v6, CD151, CA125, CEA, CTLA-4, GITR, BTLA, TGFBR2, TGFBR1, IL6R, gp130, Lewis A, Lewis Y, NGFR, MCAM, TNFR1, TNFR2, PD1, PD-L1, PD-L2, HVEM, MAGE-A, NY-ESO-1, PSMA, RANK, ROR1, ROR-2, TNFRSF4, CD40, CD137, TWEAK-R, LTPR, LIFRP, LRP5, MUC1, TCRa, TCRp, TLR7, TLR9, PTCH1, WT-1, Robol, a, Frizzled, OX40, CD79b, and Notch-1-4. The extracellular domain of the CAR interacts with and specifically binds to the tumor antigen or ligand.

A “transmembrane domain” or “spacer” is a region which links the extracellular and intracellular domains and spans part or all of the membrane. It may be borrowed from other proteins such as antibody hinge regions and CD28 respectively. For instance, the transmembrane domain may be derived from a natural protein, or may be synthetic. The transmembrane domain derived from a natural protein can be obtained from any membrane-binding or transmembrane protein. For example, a transmembrane domain of a TCR-alpha or -beta chain, a CD3-zeta chain (CD3z), CD28, CD3-epsilon (CD3e), CD45, CD4, CD5, CD8, CD9, CD16, CD22, CD33, CD37, CD64, CD80, CD86, CD134, CD137, ICOS, CD154, or a GITR can be used. A synthetic transmembrane domain may comprise hydrophobic residues such as leucine and valine. In some embodiments, a triplet of phenylalanine, tryptophan and valine may be found at each end of the synthetic transmembrane domain.

The “intracellular domain” (ICD) means any oligopeptide or polypeptide which may function as a domain that transmits a signal to cause activation or inhibition of a biological process in a cell. These domains are intracellular immune signaling motifs, for example in typical CARs may be CD3z combined with one or more T cell costimulatory domains, such CD28 or 4-1BB. An expansive and extensive set of ICDs has been created and tested in the libraries of the invention. It has been demonstrated herein that ICDs are not limited to known architectures or lineages and that unique properties may result from combinations beyond selection by function. As shown herein, a library of CARs has been constructed that contains ICDs that includes at least 1 signaling module (Modules) each of which is a signaling component. The Modules may be selected from signaling components such as CD3zeta, co-stimulatory signals such as 4-1BB, inhibitory signals such as PD1, or other signaling components such as LAT. The Modules may be selected from T cell lineages, or from other cell lineages such as B cell, NK cell, or macrophages, mast cells, or dendritic cells. Exemplary ICD domains or modules useful herein are listed in Table 1.

TABLE 1
Exemplary Intracellular Domains

| | | | | | |
|---|---|---|---|---|---|
| | CD3ζ | CD3ζ | CD3ζ | | |
| CD3ζ | (ITAM1) | (ITAM2) | (ITAM3) | CD3ε | CD3γ |

Immune activation (ITAM containing)

| | | | | | |
|---|---|---|---|---|---|
| CD3δ | CD79α | CD79β | DAP12 | FCεR1γ | K1_HHV8P |
| LMP2_EBVB9 | ENV_BLV | ENV_MMTVC | R1_RRV | VP7_AHSV | IL-2RG |
| IL-2RB | IL-7R | IL-9R | IL-2 | IL-7 | IL-9 |
| IL-21 | | | | | |

Co-stimulatory signals

| | | | | | |
|---|---|---|---|---|---|
| CD28 | DAP10 | 4-1BB | OX40 | ICOS | CDs |
| CD27 | DNAM-1 | TIM-1 | CD30 | DR3 | HVEM |
| CRTAM | 2B4 | CD84 | CD19 | CD40 | CRTAM |

Inhibitory signals

| | | | | | |
|---|---|---|---|---|---|
| CTLA4 | PD1 | TIM3 | LAG3 | BTLA | TIGIT |
| LILRB1 | LILRB2 | KIR3DL1 | KIR2DL2 | KIR2DL3 | KIR2DL5 |
| KIR3DL1 | KIR3DL2 | KIR3DL3 | SIRPα | FcRL1 | FcRL2 |
| | FcRL3 | FcRL4 | FcRL5 | FcRL6 | |

Other immune signaling components

| | | | | | |
|---|---|---|---|---|---|
| CD4 | CD8α | CD8β | LAT | FCγRIα | FCγRIIα |
| FCγRIIβ | FCγRIIIα | TLR1 | TLR2 | TLR3 | TLR4 |
| TLR5 | TLR6 | TLR7 | TLR8 | TLR9 | TLR10 |
| KIR2DL4 | PILRB | KNP46 | NKP30 | NKP44 | LY-9 |
| NKG2A | NTB-A | CRACC | CD22 | NKG2C | NKG2D |

Exemplary ICD domains or modules useful herein include but are not limited to: CD3E, CD3Z, CD3G, CD3D, CD79A, CD79B, DAP12, FCER1G, CD28, DAP10, CD137, CD134, ICOS, CD2, CD27, DNAM1, TIM1, CD30, DR3, HVEM, CRTAM, 2B4, CD84, CD19, CD40, CTLA4, PD1, TIM3, LAG3, BTLA, TIGIT, LILRB1, LILRB2, KIR3DL1, KIR3DL2, KIR2DL1, KIR2DL2, KIR2DL3, KIR2DL4, KIR2DL5, KIR3DL1, KIR3DL2, KIR3DL3, SIRPA, FCRL1, FCRL2, FCRL3, FCRL4, FCRL5, FCRL6, CD4, CD8A, CD8B, LAT, FCGR1A, FCGR2A, FCGR2B, FCGR3A, TLR1, TLR2, TLR3, TLR4, TLR5, TLR6, TLR7, TLR8, TLR9, TLR10, PILRB, NCR1, NCR2, NCR3, LY9, NKG2A, NKG2C, NKG2D, SLAMF6, SLAMF7, CD22, GITR, Human herpesvirus 8 type P K1 (K1_HHV8P), Epstein-Barr virus (strain B95-8) LMP2 (LMP2_EBVB9), Bovine leukemia virus (ENV_BLV), Mouse mammary tumor virus (strain C3H) (ENV_MMTVC), Rhesus monkey rhadinovirus H26-95 R1 (R1_RRV), African horse sickness virus (VP7_AHSV), IL-2RG, IL-2RB, IL-7R, IL-9R, IL-21R, IL-2, IL-7, IL-9, and IL-21.

In some embodiments ICD domains or modules useful herein include but are not limited to: CD3E, CD3G, CD3D, CD79A, CD79B, DAP12, FCER1G, DAP10, CD84, CD19, KIR3DL1, KIR3DL2, KIR2DL2, KIR2DL3, KIR2DL4, KIR2DL5, KIR3DL2, KIR3DL3, SIRPA, FCRL1, FCRL2, FCRL3, FCRL4, FCRL5, FCRL6, CD4, CD8A, CD8B, LAT, FCGR1A, FCGR2A, FCGR2B, FCGR3A, TLR1, TLR2, TLR3, TLR4, TLR5, TLR6, TLR7, TLR8, TLR9, TLR10, NCR1, NCR2, NCR3, LY9, NKG2C.

ICD modules include but are not limited to the following domains: an immune activation signaling domain, a co-stimulatory domain, an inhibitory signaling domain and a signaling domain from non-T cell lineage. In some embodiments, the CAR libraries have at least one non-T cell component. A non-T cell component includes for instance: CD79A, CD79B, FCER1G, CD19, CD40, KIR3DL1, KIR3DL2, KIR2DL3, KIR2DL4, KIR2DL5, KIR3DL1, KIR3DL2, KIR3DL3, SIRPA, FCRL1, FCRL2, FCRL3, FCRL4, FCRL5, FCRL6, FCGR1A, FCGR2A, FCGR2B, FCGR3A, TLR1, TLR2, TLR3, TLR4, TLR5, TLR6, TLR7, TLR8, TLR9, TLR10, PILRB, NCR1, NCR2, NCR3, NKG2A, NKG2C, NKG2D, CD22.

Each of the modules or domains disclosed herein may be combined with any other of the modules or domains disclosed herein in any combination or order. The combinations may be of 2, 3, 4, 5, 6, 7, 8, 9, or 10 or more modules or domains.

In other embodiments the CAR libraries have at least three ICDs from at least one, two or three randomly assembled modules. For instance each unique CAR may have at least three ICD modules. The identity and/or arrangement of the at least three ICD modules is different from the identity and/or arrangement of the at least three ICD modules in at least 50%, 60%, 65%, 70%, 75%, 80%, 85%, 90%, 95%, 99% or 100% of the other unique CARs in the cell library.

In one embodiment of the instant disclosure, the assembly step of the viral library involves overlap-extension polymerase chain reaction (PCR) to randomly stitch pooled Modules and Gibson Assembly (NEB) of PCR products into the lentiviral vector. In an embodiment of the instant disclosure, flexible linker sequences between each Modules may be used such that each randomly assembled CAR construct encompasses a maximum of three Modules. For example, a first designated a linker sequence (between Module 1 and Module 2) may be 10 amino acids, and this linker sequence may encode SAGGGSGGGS (SEQ ID NO: 1) and a second linker sequence (between Module 2 and Module 3) may be used and may also be 10 amino acids encoding, wherein the amino acids may be the sequence GSGSGSGSGG (SEQ ID NO: 2). In one embodiment of the instant disclosure, the linker sequences are distinct from each other in that we are excluding possibility of Module template switching during the assembly.

The CARs of the instant disclosure have ICDs of varying lengths and complexity, however, such that the ICD may be comprised of a single Module or a multitude of Modules. For example, in some embodiments, the ICD is comprised of 1, 2, 3, 4, or more Modules. Moreover, the Modules may be distinct in each ICD or the ICD may be comprise multiple copies of a Module. For example, an ICD of 3 Modules may contain only 1 unique Module which is repeated 3 times, 2 unique Modules of which 1 is repeated and 1 is distinct from the other two, or 3 unique Modules. Accordingly, the number of unique ICDs from a given set of Modules is determined by the maximum number of Modules in the ICD. For example, if the maximum number of Modules is to be 3 and a set of 3 Modules is used, the maximum number of unique ICDs will be 3+3²+3³, or 39. This equates to each Module being placed in each position of an ICD of each length. In some embodiments of the present disclosure, sets of Modules of at least 50 or more, of at least 60 or more, of at least 70 or more, of at least 75 or more, of at least 80 or more, or of at least 85 are used. In some embodiments, the length of the ICD is 1, 2, or 3 Modules in length. In one embodiment of the instant disclosure, the Modules are selected from the signaling components of Table 1. In some embodiments, the ICD has a Module comprising a signaling domain from a non-T cell lineage. In some embodiments, the ICD has a Module comprising a signaling domain from a B cell signaling domain. In some embodiments, the ICD has a Module comprising a signaling domain from a macrophage signaling domain.

In a further embodiment of the instant disclosure, a barcode is introduced into the nucleic acid sequence. A barcode is a unique nucleic acid sequence that is associated with and identifies a specific construct. Each construct or CAR in a library is associated with a single unique barcode. The barcode is a short, having a length sufficient to be distinct, nucleic acid sequence that is included in the 3′UTR of the nucleic acid sequence. When the cellular library is screened and active library members are identified, the CAR construct of such a member can be identified by sequencing the barcode.

In some aspects, highly functional novel CARs have been identified according to the invention. The CARs have, in some embodiments, an extracellular domain; a transmembrane domain; and at least a first ICD and a second ICD, wherein, the first ICD is linked to the second ICD by a linker comprising at least 10 amino acids. Nucleic acids encoding such constructs are also included in the invention.

In a preferred embodiment of the instant disclosure, unique CARs comprised of an ICD having a combination of one of the following module sets is provided: Modules CD40, CD3zITAM3, and DAP12; Modules FCER1G, 2B4, and CD3zITAM3; and Modules FCER1G, OX40, and CD3zITAM3.

In a preferred embodiment of the instant disclosure, unique CARs comprised of an ICD having a combination of one of the following module sets is provided: Modules CD40, CD3eITAM, and DAP12; Modules FCER1G, 2B4, and CD3eITAM; Modules FCER1G, OX40, and CD3zITAM3; Modules PILRB, FCER1G, and CD3zITAM3; Modules CD3zITAM3, CD3d, and CD4; and Modules CD79a, CD79aITAM, and CD4.

In some embodiments of the instant disclosure, a CAR is disclosed comprising the nucleic acid of any one of SEQ ID NO:3 (nucleic acid sequence of CAR including ICD-VAR1), 5 (nucleic acid sequence of CAR including ICD-VAR2), or 7 (nucleic acid sequence of CAR including ICD-VAR3). In some embodiments the nucleic acid comprises any of SEQ ID NO: 3, 5, or 7 without the nucleotides encoding the myc-tag. The myc tag polypeptide has the following sequence: EQKLISEEDL (SEQ ID NO: 20).

SEQ ID NO:3—Nucleic Acid sequence for a CAR encoding extracellular domain (CD8a signal peptide-Myc tag-CD19 scFv-IgG4 hinge), CD28 transmembrane domain, and ICDs (CD40-CD3eITAM-DAP12).

(SEQ ID NO: 3)
ATGGCTTTGCCTGTTACTGCGCTTCTTTTGCCTTTGGCATTGTTGCTT
CACGCCGCCAGGCCCGAGCAGAAGCTGATCAGCGAGGAGGACCTGGAC
ATACAGATGACGCAAACAACTTCCAGTCTTAGCGCTAGCCTGGGGGAT
CGAGTCACCATATCTTGCAGGGCGTCTCAAGACATTAGCAAGTATCTC
AATTGGTATCAACAGAAACCTGATGGAACAGTTAAACTTCTGATTTAC
CACACGAGTCGCCTGCACTCCGGTGTGCCCTCCAGATTCTCCGGCTC
AGGAAGTGGAACCGACTACTCTCTCACCATCTCCAACCTCGAACAAG
AAGACATAGCTACATACTTTTGCCAACAAGGTAATACCCTCCCCTATA
CCTTCGGTGGAGGCACTAAGCTGGAGATCACAGGGAGCACGTCCGGG
TCTGGCAAACCGGGGAGTGGTGAGGGGTCTACGAAGGGAGAAGTCAA
GCTTCAGGAGTCAGGACCCGGTCTTGTAGCTCCCAGCCAAAGCCTGTC
AGTTACATGCACGGTTTCCGGTGTGTCTTTGCCAGATTATGGCGTATC
TTGGATTCGCCAACCGCCTAGAAAGGGACTTGAGTGGTTGGGTGTCAT
TTGGGGATCAGAAACAACTTACTATAACAGTGCTCTTAAGTCCAGGTT
GACTATAATCAAGGACAATAGTAAGTCCCAAGTTTTTCTGAAAATGA
ATTCCCTGCAGACAGATGACACCGCTATCTACTACTGTGCCAAGCACT
ACTATTATGGGGGCTCTTATGCTATGGACTATTGGGGTCAGGGGACAT
CAGTTACTGTTTCCAGCGAAAGCAAGTATGGTCCTCCCTGCCCCCCGT
GCCCAATGTTCTGGGTGCTCGTGGTCGTAGGAGGCGTACTCGCCTGCT
ATTCATTGCTGGTTACTGTAGCCTTTATTATCTTCTGGGTCTTAATTA
AGAAGAAGGTGGCCAAAAAGCCGACAAATAAGGCCCCGCACCCTAAA
CAAGAGCCGCAAGAGATAAATTTCCCAGACGATTTGCCTGGGAGCAA
CACGGCGGCCCCGGTGCAAGAGACACTGCACGGGTGTCAACCCGTCA
CCCAAGAAGACGGAAAGGAAAGTCGGATCTCCGTCCAGGAGCGACA
GTCCGCCGGAGGGGGATCAGGAGGCGGGTCCGAAAGGCCCCCACCTG
TGCCCAATCCCGATTATGAACCAATTCGGAAAGGCCAAAGGGACCTG
TACTCAGGCCTGAATCAACGGGGCAGTGGCTCGGGCTCGGGGTCCGG
CGGATACTTTCTGGGCAGATTGGTACCAAGGGGGCGAGGTGCGGCTG
AGGCTGCCACACGGAAACAGAGGATAACGGAAACCGAGTCTCCGTAT
CAGGAACTTCAGGGACAGCGGTCCGATGTTTACAGTGACCTCAACAC
CCAAAGACCGTACTACAAGTAG

SEQ ID NO:5—Nucleic Acid sequence for a CAR sequence encoding extracellular domain (CD8a signal peptide-Myc tag-CD19 scFv-IgG4 hinge), CD28 transmembrane domain, and ICDs (FCER1G-2B4−CD3eITAM).

(SEQ ID NO: 5)
ATGGCTTTGCCTGTTACTGCGCTTCTTTTGCCTTTGGCATTGTTGCTTC
ACGCCGCCAGGCCCGAGCAGAAGCTGATCAGCGAGGAGGACCTGGAC
ATACAGATGACGCAAACAACTTCCAGTCTTAGCGCTAGCCTGGGGGA
TCGAGTCACCATATCTTGCAGGGCGTCTCAAGACATTAGCAAGTATCT
CAATTGGTATCAACAGAAACCTGATGGAACAGTTAAACTTCTGATTTA
CCACACGAGTCGCCTGCACTCCGGTGTGCCCTCCAGATTCTCCGGCTC
AGGAAGTGGAACCGACTACTCTCTCACCATCTCCAACCTCGAACAAG
AAGACATAGCTACATACTTTTGCCAACAAGGTAATACCCTCCCCTATA
CCTTCGGTGGAGGCACTAAGCTGGAGATCACAGGGAGCACGTCCGGG
TCTGGCAAACCGGGGAGTGGTGAGGGGTCTACGAAGGGAGAAGTCAA
GCTTCAGGAGTCAGGACCCGGTCTTGTAGCTCCCAGCCAAAGCCTGTC
AGTTACATGCACGGTTTCCGGTGTGTCTTTGCCAGATTATGGCGTATC
TTGGATTCGCCAACCGCCTAGAAAGGGACTTGAGTGGTTGGGTGTCAT
TTGGGGATCAGAAACAACTTACTATAACAGTGCTCTTAAGTCCAGGTT
GACTATAATCAAGGACAATAGTAAGTCCCAAGTTTTTCTGAAAATGA
ATTCCCTGCAGACAGATGACACCGCTATCTACTACTGTGCCAAGCACT
ACTATTATGGGGGCTCTTATGCTATGGACTATTGGGGTCAGGGGACAT
CAGTTACTGTTTCCAGCGAAAGCAAGTATGGTCCTCCCTGCCCCCCGT
GCCCAATGTTCTGGGTGCTCGTGGTCGTAGGAGGCGTACTCGCCTGCT
ATTCATTGCTGGTTACTGTAGCCTTTATTATCTTCTGGGTCTTAATTA
AGAGGTTGAAGATTCAGGTCCGCAAAGCGGCAATAACGAGCTACGAAA
AGTCCGACGGCGTTTATACGGGTCTTAGCACCAGGAACCAAGAGACC
TATGAAACATTGAAACATGAAAAACCCCCCCAATCCGCCGGAGGGGG
ATCAGGAGGCGGGTCCTGGAGACGGAAGAGAAAGGAGAAGCAATCC
GAAACTTCTCCCAAGGAGTTCCTCACCATTTACGAAGATGTAAAGGAC
CTGAAAACCAGACGGAATCACGAGCAAGAACAGACCTTCCCTGGCGG
CGGGTCAACTATCTACTCAATGATCCAGAGTCAAAGTTCTGCTCCAAC
TAGCCAGGAGCCGGCGTACACGCTTTACAGCCTCATTCAACCTAGCCG
CAAAAGCGGCAGCAGGAAGAGAAATCACAGTCCCTCATTCAACAGTA
CAATCTATGAGGTGATTGGCAAGTCTCAACCAAAAGCCCAGAACCCT
GCGCGACTTTCCAGGAAGGAACTCGAGAACTTCGACGTGTACTCCGG
CAGTGGCTCGGGCTCGGGGTCCGGCGGAGAAAGGCCCCCACCTGTGC
CCAATCCCGATTATGAACCAATTCGGAAAGGCCAAAGGGACCTGTAC
TCAGGCCTGAATCAACGGTAG

SEQ ID NO:7—Nucleic Acid sequence for a CAR encoding extracellular domain (CD8a signal peptide-Myc tag-CD19 scFv-IgG4 hinge), CD28 transmembrane domain, and ICDs (FCER1G-OX40-CD3zITAM3).

(SEQ ID NO: 7)
ATGGCTTTGCCTGTTACTGCGCTTCTTTTGCCTTTGGCATTGTTGCTTC
ACGCCGCCAGGCCCGAGCAGAAGCTGATCAGCGAGGAGGACCTGGAC
ATACAGATGACGCAAACAACTTCCAGTCTTAGCGCTAGCCTGGGGGA
TCGAGTCACCATATCTTGCAGGGCGTCTCAAGACATTAGCAAGTATCT
CAATTGGTATCAACAGAAACCTGATGGAACAGTTAAACTTCTGATTTA
CCACACGAGTCGCCTGCACTCCGGTGTGCCCTCCAGATTCTCCGGCTC
AGGAAGTGGAACCGACTACTCTCTCACCATCTCCAACCTCGAACAAG
AAGACATAGCTACATACTTTTGCCAACAAGGTAATACCCTCCCCTATA
CCTTCGGTGGAGGCACTAAGCTGGAGATCACAGGGAGCACGTCCGGG
TCTGGCAAACCGGGGAGTGGTGAGGGGTCTACGAAGGGAGAAGTCAA
GCTTCAGGAGTCAGGACCCGGTCTTGTAGCTCCCAGCCAAAGCCTGTC
AGTTACATGCACGGTTTCCGGTGTGTCTTTGCCAGATTATGGCGTATC
TTGGATTCGCCAACCGCCTAGAAAGGGACTTGAGTGGTTGGGTGTCAT
TTGGGGATCAGAAACAACTTACTATAACAGTGCTCTTAAGTCCAGGTT
GACTATAATCAAGGACAATAGTAAGTCCCAAGTTTTTCTGAAAATGA
ATTCCCTGCAGACAGATGACACCGCTATCTACTACTGTGCCAAGCACT
ACTATTATGGGGGCTCTTATGCTATGGACTATTGGGGTCAGGGGACAT
CAGTTACTGTTTCCAGCGAAAGCAAGTATGGTCCTCCCTGCCCCCCGT
GCCCAATGTTCTGGGTGCTCGTGGTCGTAGGAGGCGTACTCGCCTGCT
ATTCATTGCTGGTTACTGTAGCCTTTATTATCTTCTGGGTCTTAATTA
AGAGGTTGAAGATTCAGGTCCGCAAAGCGGCAATAACGAGCTACGAAA
AGTCCGACGGCGTTTATACGGGTCTTAGCACCAGGAACCAAGAGACC
TATGAAACATTGAAACATGAAAAACCCCCCCAATCCGCCGGAGGGGG
ATCAGGAGGCGGGTCCGCACTCTATCTCCTCAGACGGGATCAACGAC
TCCCGCCTGACGCCCACAAACCACCTGGTGGAGGTTCCTTTCGCACAC
CGATTCAGGAAGAACAGGCAGACGCTCATTCTACTCTCGCAAAAATC
GGCAGTGGCTCGGGCTCGGGGTCCGGCGGAGAACGACGGCGCGGCAA
GGGACATGATGGTCTGTACCAAGGTCTCTCCACAGCAACGAAGGATA
CTTACGACGCTTTGCACATGCAATAG

In some embodiments of the instant disclosure, a CAR is disclosed comprising the amino acid of any one of SEQ ID NO:4 (amino acid sequence of CAR including ICD-VAR1), 6 (amino acid sequence of CAR including ICD-VAR2), or 8 (amino acid sequence of CAR including ICD-VAR3). In some embodiments the polypeptide comprises any of SEQ ID NO: 4, 6, or 8 without the amino acids encoding the myc-tag. The myc tag polypeptide has the following sequence: EQKLISEEDL (SEQ ID NO: 20) and is underlined in the sequences.

SEQ ID NO:4—Amino Acid sequence for a CAR encoding extracellular domain (CD8a signal peptide-Myc tag-CD19 scFv-IgG4 hinge), CD28 transmembrane domain, and ICDs (CD40-CD3eITAM-DAP12).

(SEQ ID NO: 4)
MALPVTALLLPLALLLHAARPEQKLISEEDLDIQMTQTTSSLSASLGDR
VTISCRASQDISKYLNWYQQKPDGTVKLLIYHTSRLHSGVPSRFSGSGS
GTDYSLTISNLEQEDIATYFCQQGNTLPYTFGGGTKLEITGSTSGSGKP
GSGEGSTKGEVKLQESGPGLVAPSQSLSVTCTVSGVSLPDYGVSWIRQP
PRKGLEWLGVIWGSETTYYNSALKSRLTIIKDNSKSQVFLKMNSLQTDD
TAIYYCAKHYYYGGSYAMDYWGQGTSVTVSSESKYGPPCPPCPMFWVLV
VVGGVLACYSLLVTVAFIIFWVLIKKKVAKKPTNKAPHPKQEPQEINFP
DDLPGSNTAAPVQETLHGCQPVTQEDGKESRISVQERQSAGGGSGGGSE
RPPPVPNPDYEPIRKGQRDLYSGLNQRGSGSGSGSGGYFLGRLVPRGRG
AAEAATRKQRITETESPYQELQGQRSDVYSDLNTQRPYYK*

SEQ ID NO:6—Amino Acid sequence for a CAR sequence encoding extracellular domain (CD8a signal peptide-Myc tag-CD19 scFv-IgG4 hinge), CD28 transmembrane domain, and ICDs (FCER1G-2B4−CD3eITAM).

(SEQ ID NO: 6)
MALPVTALLLPLALLLHAARPEQKLISEEDLDIQMTQTTSSLSASLGDR
VTISCRASQDISKYLNWYQQKPDGTVKLLIYHTSRLHSGVPSRFSGSGS
GTDYSLTISNLEQEDIATYFCQQGNTLPYTFGGGTKLEITGSTSGSGKP
GSGEGSTKGEVKLQESGPGLVAPSQSLSVTCTVSGVSLPDYGVSWIRQP
PRKGLEWLGVIWGSETTYYNSALKSRLTIIKDNSKSQVFLKMNSLQTDD
TAIYYCAKHYYYGGSYAMDYWGQGTSVTVSSESKYGPPCPPCPMFWVLV
VVGGVLACYSLLVTVAFIIFWVLIKRLKIQVRKAAITSYEKSDGVYTGL
STRNQETYETLKHEKPPQSAGGGSGGGSWRRKRKEKQSETSPKEFLTIY
EDVKDLKTRRNHEQEQTFPGGGSTIYSMIQSQSSAPTSQEPAYTLYSLI
QPSRKSGSRKRNHSPSFNSTIYEVIGKSQPKAQNPARLSRKELENFDVY
SGSGSGSGSGGERPPPVPNPDYEPIRKGQRDLYSGLNQR*

SEQ ID NO:8—Amino Acid sequence for a CAR encoding extracellular domain (CD8a signal peptide-Myc tag-CD19 scFv-IgG4 hinge), CD28 transmembrane domain, and ICDs (FCER1G-OX40-CD3zITAM3).

(SEQ ID NO: 8)
MALPVTALLLPLALLLHAARPEQKLISEEDLDIQMTQTTSSLSASLGDRVT
ISCRASQDISKYLNWYQQKPDGTVKLLIYHTSRLHSGVPSRFSGSGSGTD
YSLTISNLEQEDIATYFCQQGNTLPYTFGGGTKLEITGSTSGSGKPGSGEG
STKGEVKLQESGPGLVAPSQSLSVTCTVSGVSLPDYGVSWIRQPPRKGLE
WLGVIWGSETTYYNSALKSRLTIIKDNSKSQVFLKMNSLQTDDTAIYYCA
KHYYYGGSYAMDYWGQGTSVTVSSESKYGPPCPPCPMFWVLVVVGGVLAC
YSLLVTVAFIIFWVLIKRLKIQVRKAAITSYEKSDGVYTGLSTRNQETYE
TLKHEKPPQSAGGGSGGGSALYLLRRDQRLPPDAHKPPGGGSFRTPIQEE
QADAHSTLAKIGSGSGSGSGGERRRGKGHDGLYQGLSTATKDTYDALHM
Human herpesvirus 8 type P K1 (K1_HHV8P)
SEQ ID NO: 9
HCQKQSDSNKTVPQQLRDYYSLHDLCTEDYTQPVDWY
Epstein-Barr virus (strain B95-8) LMP2 (LMP2_EBVB9)
SEQ ID NO: 10
HSDYQPLGTQDQSLYLGLRCCRYCCYYCLTLESEERPPTPYRNTV
Bovine leukemia virus (ENV_BLV)
SEQ ID NO: 11
APHFPEISFPPKPDSDYQALLPSAPEIYSHLSPTKPDYINLRPCP
Mouse mammary tumor virus (strain C3H) (ENV_MMTVC)
SEQ ID NO: 12
SAYDYAAIIVKRPPYVLLPVDIGD
Rhesus monkey rhadinovirus H26-95 R1 (R1_RRV)
SEQ ID NO: 13
RCNENSESSTNSYASQTSYIQPSHNQRSNTNECSRHTYRNAHQEESIEEL
PNQHTSETDSCCQLVLLEVKNVAYDGPQENTINEVMEQYDDVVVENIEQT
SYEDNVEHMDYSDTINPNFNYYSGLILEEVDEVFYNELENQYHGLILENL
DHNEYNHLNELNMIEQYDWLE
African horse sickness virus (VP7_AHSV)
SEQ ID NO: 14
EYLLLVASLADVYAAL
IL-2RG
SEQ ID NO: 15
ERTMPRIPTLKNLEDLVTEYHGNFSAWSGVSKGLAESLQPDYSERLCLVS
EIPPKGGALGEGPGASPCNQHSPYWAPPCYTLKPET
IL-2RB
SEQ ID NO: 16
NCRNTGPWLKKVLKCNTPDPSKFFSQLSSEHGGDVQKWLSSPFPSSSFSP
GGLAPEISPLEVLERDKVTQLLLQQDKVPEPASLSSNHSLTSCFTNQGYFF
FHLPDALEIEACQVYFTYDPYSEEDPDEGVAGAPTGSSPQPLQPLSGEDD
AYCTFPSRDDLLLFSPSLLGGPSPPSTAPGGSGAGEERMPPSLQERVPRDW
DPQPLGPPTPGVPDLVDFQPPPELVLREAGEEVPDAGPREGVSFPWSRPPG
QGEFRALNARLPLNTDAYLSLQELQGQDPTHLV
IL-7R
SEQ ID NO: 17
KKRIKPIVWPSLPDHKKTLEHLCKKPRKNLNVSFNPESFLDCQIHRVDDIQ
ARDEVEGFLQDTFPQQLEESEKQRLGGDVQSPNCPSEDVVITPESFGRDSS
LTCLAGNVSACDAPILSSSRSLDCRESGKNGPHVYQDLLLSLGTTNSTLPP
PFSLQSGILTLNPVAQGQPILTSLGSNQEEAYVTMSSFYQNQ
IL-9R
SEQ ID NO: 18
KLSPRVKRIFYQNVPSPAMFFQPLYSVHNGNFQTWMGAHGAGVLLSQD
CAGTPQGALEPCVQEATALLTCGPARPWKSVALEEEQEGPGTRLPGNLSS
EDVLPAGCTEWRVQTLAYLPQEDWAPTSLTRPAPPDSEGSRSSSSSSSSN
NNNYCALGCYGGWHLSALPGNTQSSGPIPALACGLSCDHQGLETQQGV
AWVLAGHCQRPGLHEDLQGMLLPSVLSKARSWTF
IL-21R
SEQ ID NO: 19
SLKTHPLWRLWKKIWAVPSPERFFMPLYKGCSGDFKKWVGAPFTGSSLE
LGPWSPEVPSTLEVYSCHPPRSPAKRLQLTELQEPAELVESDGVPKPSFWP
TAQNSGGSAYSEERDRPYGLVSIDTVTVLDAEGPCTWPCSCEDDGYPAL
DLDAGLEPSPGLEDPLLDAGTTVLSCGCVSAGSPGLGGPLGSLLDRLKPP
LADGEDWAGGLPWGGRSPGGVSESEAGSPLAGLDMDTFDSGFVGSDCS
SPVECDFTSPGDEGPPRSYLRQWVVIPPPLSSPGPQAS

In some embodiments, a CAR may be comprised of a nucleic acid sequence with a percent identity of varying amounts to SEQ ID NO: 3, 5, or 7 such as 70%, 80%, 85%, 90%, 95%, or 99%. In other embodiments, a CAR may be comprised of an amino acid sequence with a percent identity of varying amounts to SEQ ID NO: 4, 6, or 8 such as 70%, 80%, 85%, 90%, 95%, or 99%.

### EXAMPLES

### Example 1: Large CAR Libraries Require Additional Intracellular Signaling Domains

### Introduction

Experiments for this have been conducted, devising ways to construct the library, perform selections, and sequence the library. Intracellular domain variants have also been found that, at least preliminarily when tested for function, are distinct from, and potentially superior to, previously described earlier version CARs.

Creation of an Immune Receptor Intracellular Domain Library Linked to an Anti-CD19 scFV

It was shown that the size of a CAR library and potential efficacy and range of activities of CAR-T cell therapeutics, and of T cells in general, is not limited to the small number of naturally occurring and engineered Module combinations that have been vetted even as proofs of concept. To explore this hypothesis, a library of CARs was curated which exploits Modules using a wide array of potential signaling inputs, including any combination of immune-relevant signaling inputs to produce desirable functional phenotypes.

Therefore a curated list of 85 immune receptor signaling domains (many from T cells, but also including domains from B cells, macrophages, and other immune cells) was prepared. When a signaling domain had multiple distinct subunits (such as CD3z), both their constitutive subunits as well as the whole domain were included (FIG. 1B).

To create this library, a PCR-based strategy to combinatorically shuffle every possible 2- and 3-intracellular domain combination in the context of an anti-CD19 CAR was used. Each construct was additionally tagged with a unique barcode to enable tracking via sequencing. The initial CAR library consisted of approximately 500,000 sequences. Modules are being continually added to this initial library, including such additional sequences as virally encoded ITAMs (which may show distinct activity to those found in the human genome), and the intracellular domains of immune-specific growth factor receptors such as IL-2R-beta and IL-2R-gamma (FIGS. 2A-C).

To create a T cell library from the collection of sequences, the assembled ICD collection is then inserted into a standard lentiviral transfer vector, generate lentiviruses on large scale, and then transduce T-cell T cells at low multiplicity of infection (MOI) to minimize the ability for multiple viruses to infect one cell.

### Selection of a Library Based Upon Immune Cell Function

The identification of active CAR sequences requires a method to sort for T cells expressing receptors that confer some type of signaling output or cellular phenotype. Further, since library-based paradigms often require identifying active variants that are rare relative to the rest of the library, this requires techniques that are sensitive, robust, and non-damaging to viable cells to enable their subsequent recovery.

A wide array of potential assays has been established to achieve these goals, including surface expression of known T cell activation markers such as CD69, production of soluble factors of T cell function such as IL-2 or IFN-γ (via commercially available secrete and capture kits), cytotoxicity (via upregulation of the degranulation marker CD107a), and proliferation. In principle, any function or cell phenotype of interest can be sued so long as there is a condition that can be linked to cell sorting. These protocols have been optimized and are able to enrich active CAR sequences (such as the canonical 4-1BB-CD3z CAR) from a background of sequences containing inactive signaling domains, and have since conducted selections to identify active CAR sequences.

### Sequencing

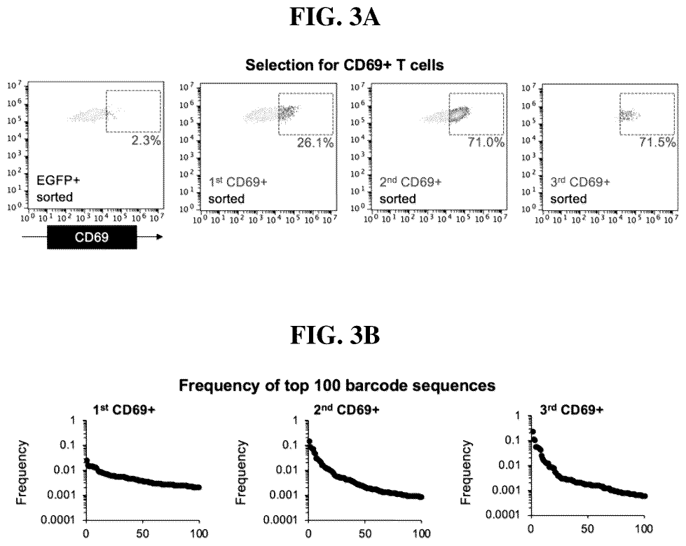

While next-generation sequencing has enabled a wide range of library-based studies, this project presents unique challenges. Since many of the ICD combinations are quite long (over 2 kilobases), Illumina-based sequencing is not suited to this application. Conversely, long amplicon based approaches such as PacBio sequencing do not provide the sequencing read depth or sufficient quantification between amplicons of different sizes to fully quantitate the library (FIGS. 3A-3B).

Therefore, approaches have been combined to enable characterization and quantitation of the library: PacBio sequencing of library samples is used to create a ‘look-up table’ to establish connectivity between combinations of ICDs and associated 3′ barcode sequences, and Illumina is used to quantitatively deep sequence the library barcodes through each round of selection.

### Initial Experiment Results

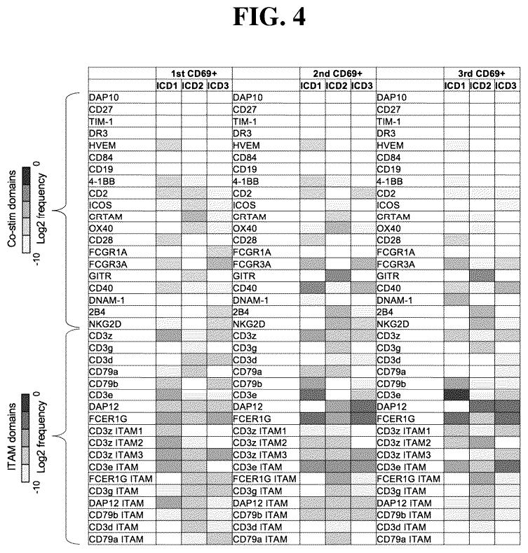

The steps described herein have been successfully combined in Jurkat cells, a T cell lymphoma line. Sorting was done primarily for CD69 upregulation upon exposure to CD19 antigen, although also combined sorting for CD69+PD1− cells (in an attempt to find cells that could activate while limiting the exhausted T cell phenotype). After iterating activation, staining, and sorting for multiple cycles, an enriched population was identified that upon sequencing demonstrated itself to be hundreds to thousands of unique CAR sequences (FIGS. 3A-3B). These sequences show a range of ICD usage and potential positional preferences (FIG. 4). Notably, essentially all sequences contain an ITAM activation domain, a strong indicator that they are all active.

This experiment is being performed in primary CD4+ and CD8+ T cells—it is possible that primary cells will have an expanded range of functional plasticity, creating different or more stringent selection criteria.

### Characterization of Selected CAR Variant Sequences

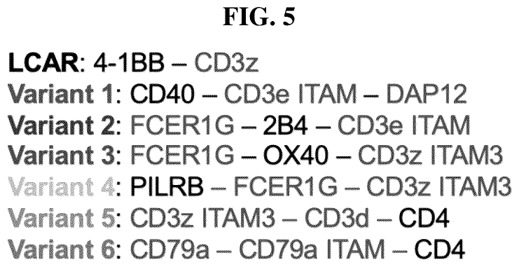

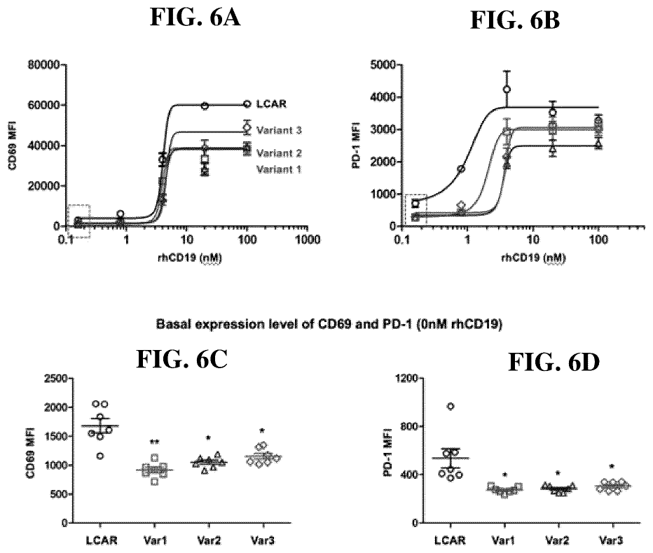

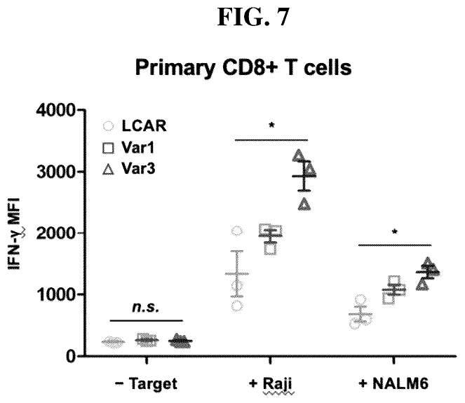

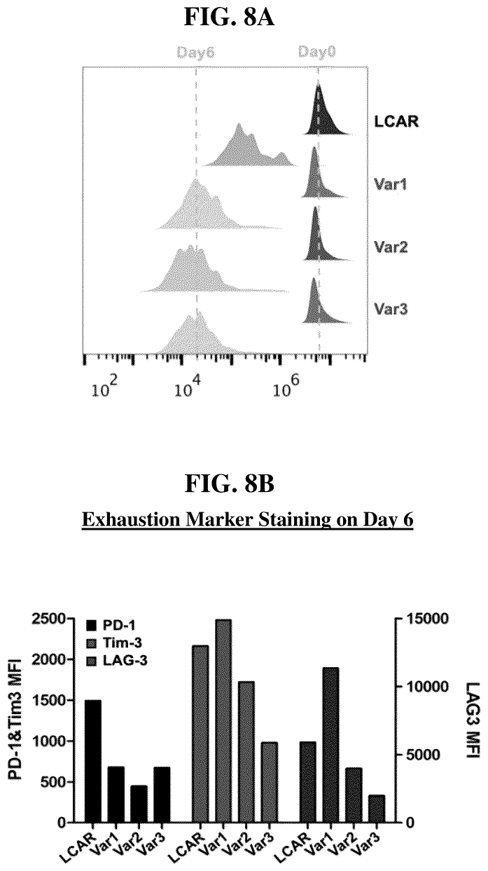

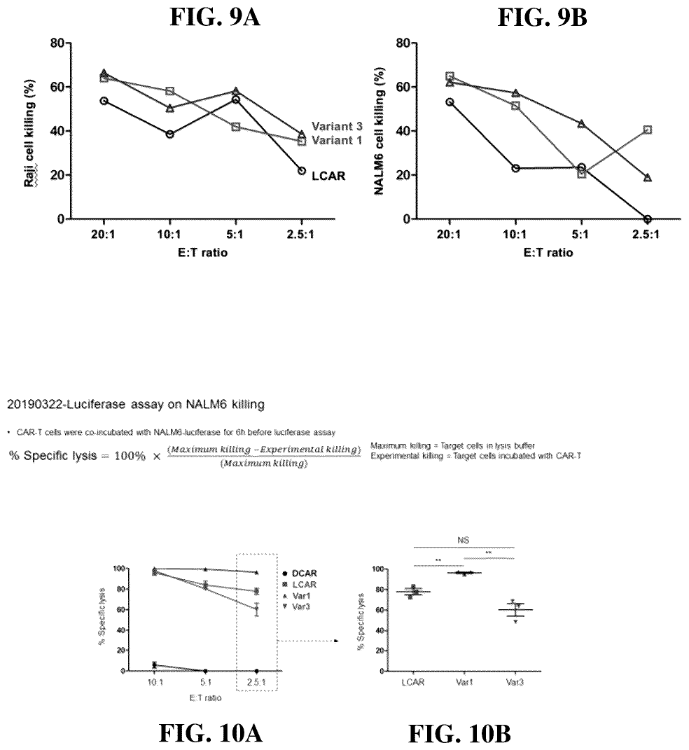

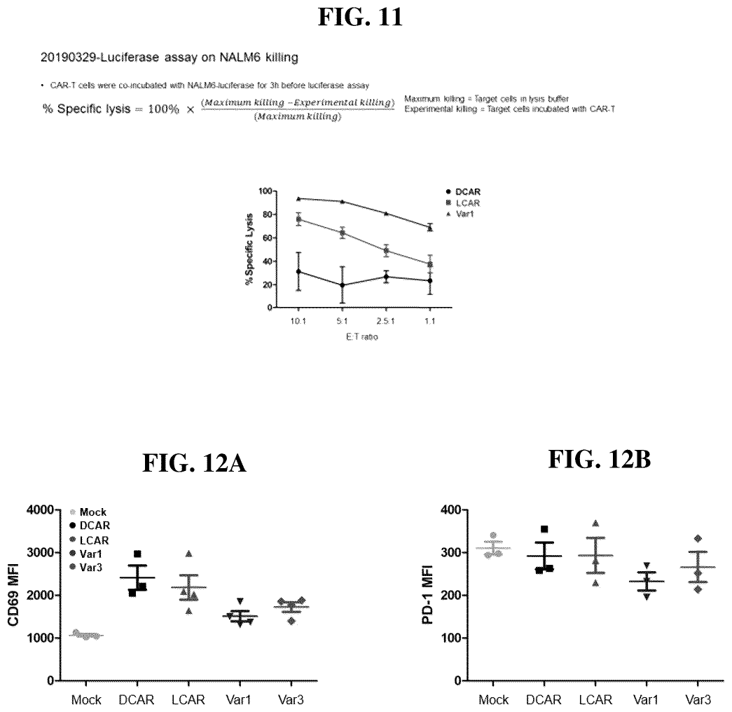

In addition to continuing the selections, some of the most distinct enriched ‘hits’ from the initial selection study have been analyzed. Six sequences have been examined, and three have primarily been focused on (FIG. 5). Excitingly, multiple differences were observed between the sequences and the 2nd generation CAR comparator in both Jurkats and primary T cells. While experiments are still being conducted (including key in vivo validations in mice), several results have been observed. As shown in FIGS. 6A-6D, changes in surface levels of PD1 and CD69 have been observed, both at resting state and after activation. There were increases in IFN-y production (FIG. 7) and increased proliferation in primary CD8+ T cells (FIG. 8A). There was also altered expression of canonical T cell exhaustion markers (including PD-1, LAG-3, and TIM-3; FIG. 8A). Notably here, these markers do not change in lockstep for various CAR variants, meaning for some, these markers are no longer correlated (FIGS. 8A-8B). Also, as shown in FIGS. 9A-9B, there were intriguing, albeit preliminary, improvements observed for in vitro cell killing of B cell cancer lines.

### Example 2: ICDs can Decrease Basal Signaling States

The functional attributes of Var1 and Var3 were further characterized in a series of experiments. It has been shown that CARs with higher basal signaling states are associated with lower efficacy in the clearance of tumors in vivo. The basal signaling states of the Var1, Var3, a negative control (DCAR) and a positive control (LCAR) were tested. The results were shown in FIGS. 12A-12B. A comparison of basal signaling states based on CD69 and PD-1 level in unstimulated CD8+ T cells from 3-4 different donors is presented. Var1 (CD40, CD3e ITAM, and DAP12) shows a lower degree of basal signaling assessed by activation markers, CD69 (FIG. 12A) and PD-1 (FIG. 12B), over other CARs. Mock: Cells treated identically to CAR-transduced cells but that do not express a CAR construct; LCAR chimeric antigen receptor encoding the 4-1BB and CD3Z signaling domains as currently used in the clinic); DCAR, an LCAR construct with each Tyrosine that is phosphorylated via signaling mutated to Phenylalanine, creating a signaling-inactive CAR variant; and Var3 (FCER1G-OX40-CD3z ITAM 3).

### Example 3: ICDs can Increase Anti-Tumor Cytokines and Chemokines

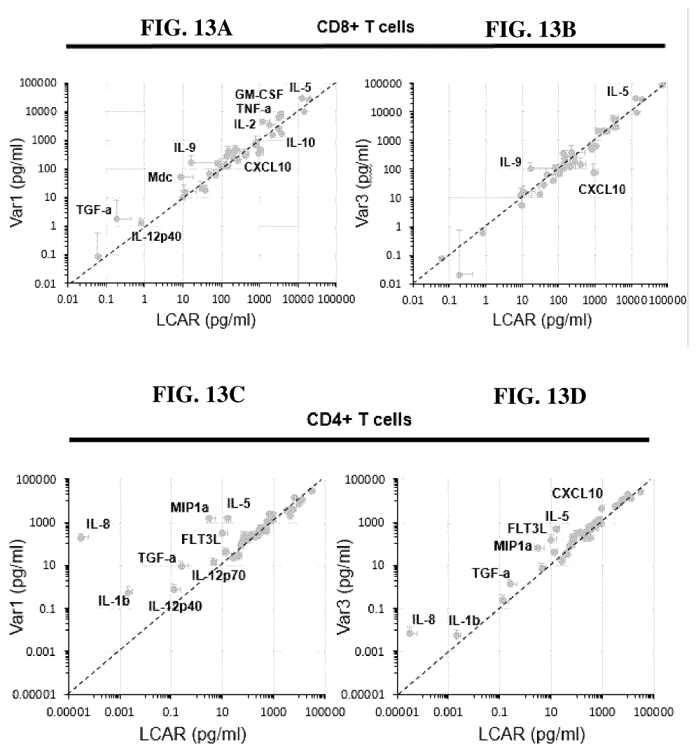

In order to determine the immunogenicity of the variants, the influence of these constructs on anti-tumor cytokines and chemokines was analyzed. CAR-T cells were co-cultured with NALM6 cells at effector:target ratio of 1:1 for 24 h prior to 41-plex Luminex assay and showed differential cytokine and chemokine expression by Var1 and Var3 compared to LCAR in CD4+ or CD8+ T cells (FIGS. 13A-13D). Var1 and Var3 show elevated levels of secreted anti-tumor cytokines and chemokines over LCAR, such as MIP1a, FLT3L, IL-12p70, TNF-a, GM-CSF, and IL-2 (FIGS. 13A-13D).

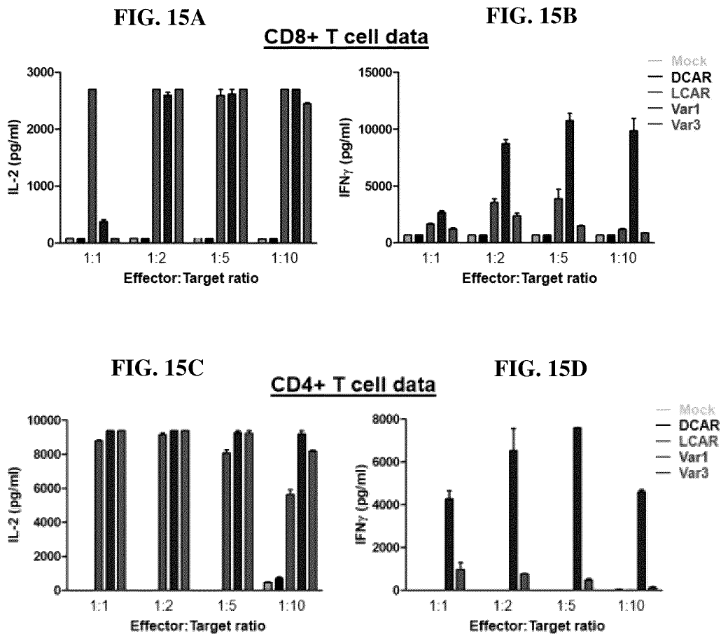

CAR-T cells were challenged with NALM6 cells at designated effector:target ratio (FIGS. 15A-15D) and showed elevated levels of IL-2 and IFN-γ levels, two major anti-tumor cytokines of T cells, of CD8+ or CD4+. IFNγ secretion level of Var1 is significantly higher than that of other types of CARs across different amount of tumor burden in both CD4+ and CD8+ T cells. CAR-T cells were co-cultured with NALM6 cells for 24 h prior to ELISA assay.

### Example 4: ICDs can Increase CAR-T Killing Capacity

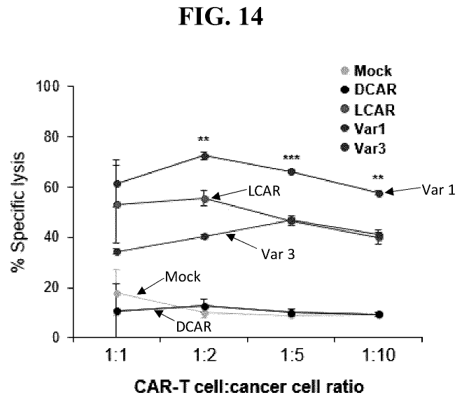

The variants descried herein were shown to be effective in CAR-T cell killing. CAR-T cells were co-cultured with NALM6 cells at designated effector:target ratio for 24 h prior to luciferase assay (FIG. 14) and show the killing capacity of CAR-T cells at increasing effector:target ratios. Var1 in CD8+ T cells shows results at controlling high tumor burden compared to that of LCAR.

### Example 5: ICDs can Increase CAR-T Cell Proliferation Rates

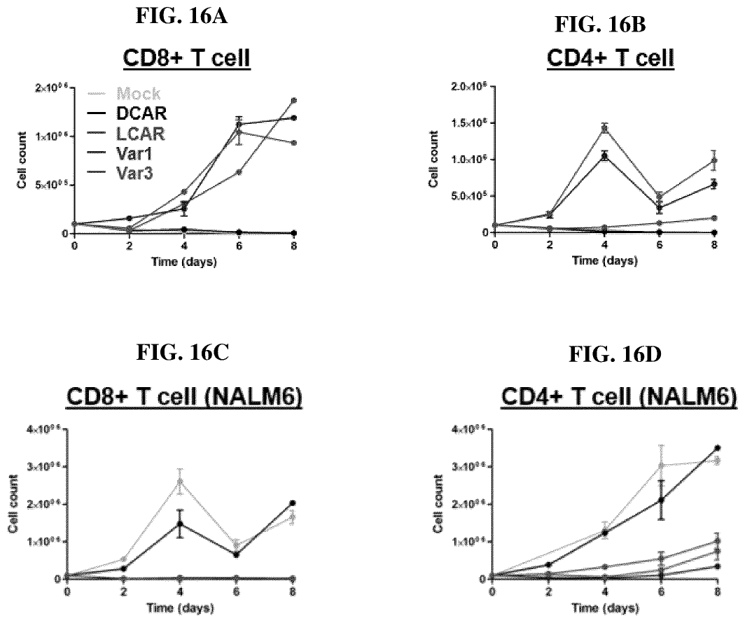

Proliferation and tumor control are important properties for therapeutically effective CAR-T cells. The new constructs were tested to determine the impact on CAR-T cell proliferation rates. These were assessed by counting absolute cell numbers in the culture across 8 days after repetitive challenge with NALM6 cells every 48 h to maintain effector:target ratio of 1:2 (FIGS. 16A-16D). Significantly, Var1 and Var3 in CD4+ T cells showed better proliferation and tumor control compared to those of the positive control LCAR (FIGS. 16A-16D).

### Example 6: ICDs can Effect CAR-T Cell Exhaustion Markers

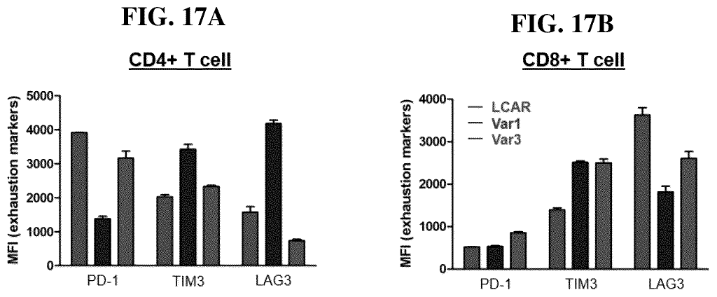

Exhaustion markers on CD4+ or CD8+ CAR-T cells were stained at day 10 of repetitive challenge assay. CAR-T cells were co-cultured with NALM6 cells and effector:target ratio of 1:2 was maintained throughout by adding target cells every 48 h (FIGS. 17A-17B). High expression of PD-1, TIM3, and LAG3 exhaustion markers in dysfunctional T cells is a hallmark of exhausted T cells (FIGS. 17A-17B). Under these experimental conditions, there was variability in differential expression levels of exhaustion markers among CAR-T cells, Var 1, and Var 3. Other assays may provide a clearer picture on the exhaustion of the tested CAR-T cells.

### OTHER EMBODIMENTS

Paragraph 1 A CAR, comprising: an extracellular domain; a transmembrane domain; and at least a first intracellular domain (ICD) and a second ICD, wherein, the first ICD is linked to the second ICD by a linker comprising at least 10 amino acids.
Paragraph 2 A CAR, comprising: an extracellular domain; a transmembrane domain; and at least a first intracellular domain (ICD), wherein the ICD comprises at least three linked modules that are CD40, CD3eITAM, and DAP12.
Paragraph 3 A CAR, comprising: an extracellular domain; a transmembrane domain; and at least a first intracellular domain (ICD), wherein the ICD comprises at least modules that are FCER1G, 2B4 and CD3eITAM.
Paragraph 4 A CAR, comprising: an extracellular domain; a transmembrane domain; and at least a first intracellular domain (ICD), wherein the ICD comprises at least three linked modules that are FCER1G, OX40, and CD3zITAM3.
Paragraph 5 A CAR, comprising: an extracellular domain; a transmembrane domain; and at least a first intracellular domain (ICD), wherein the ICD comprises at least three linked modules that are CD40, CD3zITAM3, and DAP12.
Paragraph 6 A CAR, comprising: an extracellular domain; a transmembrane domain; and at least a first intracellular domain (ICD), wherein the ICD comprises at least three linked modules that are FCER1G, 2B4, and CD3zITAM3.
Paragraph 7 A CAR, comprising: an extracellular domain; a transmembrane domain; and at least a first intracellular domain (ICD), wherein the ICD comprises at least three linked modules that are FCER1G, OX40, and CD3zITAM.
Paragraph 8 A CAR, comprising: an extracellular domain; a transmembrane domain; and at least a first intracellular domain (ICD), wherein the ICD comprises at least three linked modules that are PILRB, FCER1G, and CD3zITAM3.
Paragraph 9 A CAR, comprising: an extracellular domain; a transmembrane domain; and at least a first intracellular domain (ICD), wherein the ICD comprises at least three linked modules that are CD3zITAM3, CD3d, and CD4.
Paragraph 10 A CAR, comprising: an extracellular domain; a transmembrane domain; and at least a first intracellular domain (ICD), wherein the ICD comprises at least three linked modules that are CD79a, CD79aITAM, and CD4.
Paragraph 11. A CAR of any one of paragraphs 1-10, wherein the extracellular domain is a CD8a signal peptide and CD19 scFv-IgG4 hinge).
Paragraph 12. A CAR of any one of paragraphs 1-10, wherein the transmembrane domain is CD28.
Paragraph 13. A nucleic acid, comprising a coding region that encodes any of the CARs of the above paragraphs.
Paragraph 14. A nucleic acid wherein the nucleic acid sequence has at least 70% sequence identity to (SEQ ID NO:3).
Paragraph 15. A nucleic acid wherein the nucleic acid sequence has at least 80% sequence identity to (SEQ ID NO:3).
Paragraph 16. A nucleic acid wherein the nucleic acid sequence has at least 70% sequence identity to (SEQ ID NO:3).
Paragraph 17. A nucleic acid wherein the nucleic acid sequence has at least 85% sequence identity to (SEQ ID NO:3).
Paragraph 18. A nucleic acid wherein the nucleic acid sequence has at least 90% sequence identity to (SEQ ID NO:3).
Paragraph 19. A nucleic acid wherein the nucleic acid sequence has at least 95% sequence identity to (SEQ ID NO:3).
Paragraph 20. A nucleic acid wherein the nucleic acid sequence has at least 99% sequence identity to (SEQ ID NO:3).
Paragraph 21. A nucleic acid wherein the nucleic acid sequence has at least 70% sequence identity to (SEQ ID NO:5).
Paragraph 22. A nucleic acid wherein the nucleic acid sequence has at least 80% sequence identity to (SEQ ID NO:5).
Paragraph 23. A nucleic acid wherein the nucleic acid sequence has at least 70% sequence identity to (SEQ ID NO:5).
Paragraph 24. A nucleic acid wherein the nucleic acid sequence has at least 85% sequence identity to (SEQ ID NO:5).
Paragraph 25 A nucleic acid wherein the nucleic acid sequence has at least 90% sequence identity to (SEQ ID NO:5).
Paragraph 26. A nucleic acid wherein the nucleic acid sequence has at least 95% sequence identity to (SEQ ID NO:5).
Paragraph 27 A nucleic acid wherein the nucleic acid sequence has at least 99% sequence identity to (SEQ ID NO:5).
Paragraph 28. A nucleic acid wherein the nucleic acid sequence has at least 70% sequence identity to (SEQ ID NO:7).
Paragraph 29. A nucleic acid wherein the nucleic acid sequence has at least 80% sequence identity to (SEQ ID NO:7).
Paragraph 30. A nucleic acid wherein the nucleic acid sequence has at least 70% sequence identity to (SEQ ID NO:7).
Paragraph 31. A nucleic acid wherein the nucleic acid sequence has at least 85% sequence identity to (SEQ ID NO:7).
Paragraph 32. A nucleic acid wherein the nucleic acid sequence has at least 90% sequence identity to (SEQ ID NO:7).
Paragraph 33. A nucleic acid wherein the nucleic acid sequence has at least 95% sequence identity to (SEQ ID NO:7).
Paragraph 34. A nucleic acid wherein the nucleic acid sequence has at least 99% sequence identity to (SEQ ID NO:7).
Paragraph 35 A polypeptide, comprising an amino acid sequence translated from the coding region that encodes any of the CARs of the above paragraphs.
Paragraph 36 A polypeptide having an amino acid sequence, wherein the amino acid sequence has at least 70% sequence identity to (SEQ ID NO:4).
Paragraph 37 A polypeptide having an amino acid sequence, wherein the amino acid sequence has at least 80% sequence identity to (SEQ ID NO:4).
Paragraph 38 A polypeptide having an amino acid sequence, wherein the amino acid sequence has at least 70% sequence identity to (SEQ ID NO:4).
Paragraph 39 A polypeptide having an amino acid sequence, wherein the amino acid sequence has at least 85% sequence identity to (SEQ ID NO:4).
Paragraph 40 A polypeptide having an amino acid sequence, wherein the amino acid sequence has at least 90% sequence identity to (SEQ ID NO:4).
Paragraph 41 A polypeptide having an amino acid sequence, wherein the amino acid sequence has at least 95% sequence identity to (SEQ ID NO:4).
Paragraph 42 A polypeptide having an amino acid sequence, wherein the amino acid sequence has at least 99% sequence identity to (SEQ ID NO:4).
Paragraph 43 A polypeptide having an amino acid sequence, wherein the amino acid sequence has at least 70% sequence identity to (SEQ ID NO:6).
Paragraph 44 A polypeptide having an amino acid sequence, wherein the amino acid sequence has at least 80% sequence identity to (SEQ ID NO:6).
Paragraph 45 A polypeptide having an amino acid sequence, wherein in the amino acid sequence has at least 70% sequence identity to (SEQ ID NO:6).
Paragraph 46 A polypeptide having an amino acid sequence, wherein the amino acid sequence has at least 85% sequence identity to (SEQ ID NO:6).
Paragraph 47 A polypeptide having an amino acid sequence, wherein the amino acid sequence has at least 90% sequence identity to (SEQ ID NO:6).
Paragraph 48 A polypeptide having an amino acid sequence, wherein the amino acid sequence has at least 95% sequence identity to (SEQ ID NO:6).
Paragraph 49 A polypeptide having an amino acid sequence, wherein the amino acid sequence has at least 99% sequence identity to (SEQ ID NO:6).
Paragraph 50 A polypeptide having an amino acid sequence, wherein the amino acid sequence has at least 70% sequence identity to (SEQ ID NO:8).
Paragraph 51 A polypeptide having an amino acid sequence, wherein the amino acid sequence has at least 80% sequence identity to (SEQ ID NO:8).
Paragraph 52 A polypeptide having an amino acid sequence, wherein the amino acid sequence has at least 70% sequence identity to (SEQ ID NO:8).
Paragraph 53 A polypeptide having an amino acid sequence, wherein the amino acid sequence has at least 85% sequence identity to (SEQ ID NO:8).
Paragraph 54 A polypeptide having an amino acid sequence, wherein the amino acid sequence has at least 90% sequence identity to (SEQ ID NO:8).
Paragraph 55 A polypeptide having an amino acid sequence, wherein the amino acid sequence has at least 95% sequence identity to (SEQ ID NO:8).
Paragraph 56 A polypeptide having an amino acid sequence, wherein the amino acid sequence has at least 99% sequence identity to (SEQ ID NO:8).
Paragraph 57 A nucleic acid of any CAR of the above paragraphs, wherein the nucleic acid further comprises a 18-nucleotide long barcode in a 3′ untranslated region (3′-UTR).
Paragraph 58 A nucleic acid of any CAR of the above paragraphs, wherein the nucleic acid does not include a sequence encoding a myc-tag.
Paragraph 59 A polypeptide of any CAR of the above paragraphs, wherein the polypeptide does not include a myc-tag.
Paragraph 60 A nucleic acid or polypeptide of paragraph 58 or 59 wherein the myc-tag has the following sequence: EQKLISEEDL (SEQ ID NO: 20).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1-39. (canceled)

40. A CAR, comprising:
i) an extracellular domain;
ii) a transmembrane domain; and
iii) at least a first intracellular domain (ICD) and a second ICD,
wherein, the first ICD is linked to the second ICD by a linker comprising at least 10 amino acids.

41. (canceled)

42. A nucleic acid, comprising a coding region that encodes the CAR of claim 40, wherein the nucleic acid sequence has at least 70%, at least 80%, at least 85%, at least 90%, or at least 99% sequence identity to any one of SEQ ID NOs:3, 5, or 7 or to any one of SEQ ID NOs:3, 5, or 7 without a nucleic acid sequence encoding a myc-tag.

43-45. (canceled)

46. A polypeptide, comprising an amino acid sequence wherein the amino acid sequence has at least 70%, at least 80%, at least 85%, at least 90%, or at least 99% sequence identity to any one of SEQ ID NOs:4, 6, or 8 or to any one of SEQ ID NOs:4, 6, or 8 without an amino acid sequence comprising a myc-tag.

47-48. (canceled)

49. The nucleic acid of claim 42, wherein the nucleic acid further comprises a 18-nucleotide long barcode in a 3′ untranslated region (3′-UTR).

50. A CAR, comprising: an extracellular domain; a transmembrane domain; and an intracellular domain (ICD) comprised of three linked modules, wherein the three linked modules are any one of CD40-CD3zITAM3-DAP12, FCER1G-2B4-CD3zITAM3, or FCER1G-OX40-CD3zITAM.

51-52. (canceled)

53. A CAR, comprising: an extracellular domain; a transmembrane domain; and an intracellular domain (ICD) comprised of three linked modules, wherein the three linked modules are any one of CD40-CD3eITAM-DAP12 FCER1G-2B4-CD3eITAM, FCER1G-OX40-CD3zITAM3, PILRB-FCER1G-CD3zITAM3, CD3zITAM3-CD3d-CD4, or CD79a-CD79aITAM-CD4.

54-58. (canceled)

59. A CAR, comprising: an extracellular domain; a transmembrane domain; and at least a first intracellular domain (ICD) and a second ICD, wherein, the first ICD is linked to the second ICD by a linker comprising at least 10 amino acids.

60. The CAR of claim 59, wherein the ICD comprises at least three linked modules that are CD40, CD3eITAM, and DAP12.

61. The CAR of claim 59, wherein the ICD comprises at least modules that are FCER1G, 2B4 and CD3eITAM.

62. The CAR of claim 59, wherein the ICD comprises at least modules that are FCER1G, 2B4 and CD3eITAM.

63. The CAR of claim 59, wherein the ICD comprises at least three linked modules that are FCER1G, OX40, and CD3zITAM3.

64. The CAR of claim 59, wherein the ICD comprises at least three linked modules that are CD40, CD3zITAM3, and DAP12.

65. The CAR of claim 59, wherein the ICD comprises at least three linked modules that are FCER1G, 2B4, and CD3zITAM3.

66. The CAR of claim 59, wherein the ICD comprises at least three linked modules that are FCER1G, OX40, and CD3zITAM.

67. The CAR of claim 59, wherein the ICD comprises at least three linked modules that are PILRB, FCER1G, and CD3zITAM3.

68. The CAR of claim 59, wherein the ICD comprises at least three linked modules that are CD3zITAM3, CD3d, and CD4.

69. The CAR of claim 59, wherein the ICD comprises at least three linked modules that are CD79a, CD79aITAM, and CD4.

70. The CAR of claim 59, wherein the extracellular domain is a CD8a signal peptide and CD19 scFv-IgG4 hinge.

71. The CAR of claim 59, wherein the transmembrane domain is CD28.
